(19) United States
(12) Patent Application Publication
(10) Pub. No.: US 2014/0309491 A1
(43) Pub. Date: Oct. 16, 2014

(54) ENDOSCOPE SYSTEM WITH PROCESSING APPARATUS

(71) Applicant: FUJIFILM Corporation, Tokyo (JP)

(72) Inventor: Hiroyuki KARASAWA, Ashigarakami-gun (JP)

(73) Assignee: FUJIFILM Corporation, Tokyo (JP)

(21) Appl. No.: 14/252,114

(22) Filed: Apr. 14, 2014

(30) Foreign Application Priority Data
Apr. 15, 2013 (JP) 2013-084862

(51) Int. Cl. A61B 1/00; A61B 1/06; A61B 1/05

(52) U.S. Cl. CPC A61B 1/00124; A61B 1/00016; A61B 1/05; A61B 1/00009; A61B 1/00029; A61B 1/00126; A61B 1/0669
USPC 600/103

(57) ABSTRACT

An endoscope system includes an endoscope and a processing apparatus. A communication connector for the endoscope includes a plug unit and a finger grip. The plug unit includes a connector housing and a shielding device. A first near field communication interface for NFC communication is incorporated in the connector housing. In the processing apparatus, a receiving cavity is formed in a receptacle connector, which also has a socket housing and a shielding device. A second near field communication interface is incorporated in the socket housing. In case the communication connector is coupled with the receptacle connector connectively, the second near field communication interface is positioned at the first near field communication interface at a short distance.

### BACKGROUND OF THE INVENTION

1. Field of the Invention

The present invention relates to an endoscope system with a processing apparatus. More particularly, the present invention relates to an endoscope system in which radio communication is utilized with a processing apparatus, and electric noise can be prevented reliably.

2. Description Related to the Prior Art

An electronic endoscope is widespread for diagnosis of an object in a body in the medical field, industrial field and the like. The endoscope includes a grip handle and an elongated tube for entry in the body. A tip device of the elongated tube contains an image sensor, for example, CCD image sensor. A processing apparatus is connected with the endoscope by a connector. An image signal obtained by the image sensor is transmitted to the processing apparatus, which forms an endoscopic image and drives a monitor display panel to display the endoscopic image.

An endoscope system with electrical connection in a non-contact manner to the processing apparatus has been suggested in consideration of suitability for washing or cleaning the endoscope. For example, JP-A 7-327922, U.S. Pub. No. 2004/104,999 (corresponding to JP-A 2004-159833), U.S. Pub. No. 2009/247,828 (corresponding to JP-A 2009-247407), JP-A 2003-190081 and JP-A 2003-250758 disclose the use of radio communication in the endoscope system. An image signal output by an image sensor in the endoscope is processed to generate radio waves, which is received by the processing apparatus to drive a display panel to display the endoscopic image after the image processing. In general, the radio communication according to wireless LAN standards is widely used in the endoscope system.

According to an example of the endoscope system disclosed in JP-A 2003-190081, a large area of the endoscope and the processing apparatus is in a shielded structure. An end face of an opening for connection of the endoscope and the processing apparatus is an open structure without shield. While the endoscope is connected with the processing apparatus, the entirety of those is a shielded construction, in which external electromagnetic waves are blocked.

JP-A 2003-250758 discloses the endoscope system in which NFC communication (near field wireless communication) is utilized between the endoscope and the processing apparatus, to display the endoscopic image on a display panel connected with the processing apparatus. The NFC communication is a type of radio communication of a short distance of transmission, and widely used today, for example, in an IC card, cellular telephone and the like.

In the radio communication according to the wireless LAN standards, it is necessary initially to set up conditions of the SSID (service set identifier), user verification, data encryption and the like. Manipulation of the endoscope system for the setup is very complicated and laborious. Also, setting of a communication channel is required for each one of combinations of the endoscope and the processing apparatus for the purpose of discrete use of a plurality of the endoscope and a plurality of the processing apparatus. However, a wavelength range of radio waves of the wireless LAN standards is considerably narrow, so that the number of available communication channels is limited. It is likely that the same communication channel is incidentally used in another external device of the wireless LAN standards located nearby. The wireless LAN standards are not preferable due to electric noise according to crosstalk and a problem of security in the communication.

In the endoscope system of JP-A 2003-190081, the endoscope and the processing apparatus are in the shielded construction, to reduce possibility of crosstalk with external signals. However, a manufacturing cost will be high due to the entirety of the endoscope and the processing apparatus in the shielded construction.

In the endoscope system of JP-A 2003-250758, the NFC communication is used without requiring the complicated setup of the wireless LAN standards. However, the endoscope is not physically connected with the processing apparatus, but electrically connected in the non-contact manner. Should a device of the same standards of the NFC communication be located at a short distance, there occurs a problem of electric noise due to crosstalk in the same manner as the wireless LAN standards.

### SUMMARY OF THE INVENTION

In view of the foregoing problems, an object of the present invention is to provide an endoscope system in which radio communication is utilized with a processing apparatus, and electric noise can be prevented reliably.

In order to achieve the above and other objects and advantages of this invention, an endoscope system is provided, and includes an endoscope having a tip device for entry in a body cavity, and an imaging unit for receiving light from an object in the body cavity through the tip device to output an image signal, and a processing apparatus for forming an endoscopic image according to the image signal from the imaging unit. The endoscope system includes a first connector disposed on a proximal end side of the endoscope. A second connector is disposed on the processing apparatus, and coupled with the first connector. A first near field communication interface is disposed in the first connector, for wirelessly transmitting the image signal from the imaging unit, and wirelessly receiving a control signal for controlling the imaging unit. A second near field communication interface is disposed in the second connector, and positioned at a short distance to the first near field communication interface upon setting of the first and second connectors in a connected state. A connection detection device detects the connected state between the first and second connectors. An interface controller controls the first and second near field communication interfaces for near field wireless communication in case the connection detection device detects the connected state between the first and second connectors.

Preferably, the first and second near field communication interfaces perform the near field wireless communication upon being enabled and also located at a short distance for communicability.

Preferably, incase the connection detection device detects the connected state between the first and second connectors, the interface controller enables the first near field communication interface.

Preferably, in case the connection detection device detects the connected state between the first and second connectors, the interface controller enables the second near field communication interface.

Preferably, furthermore, a contact terminal is disposed in the first and second connectors, for supplying the endoscope with power from the processing apparatus upon setting of the first and second connectors in the connected state. The interface controller enables the first near field communication interface after starting supplying the endoscope with power through the contact terminal.

Preferably, furthermore, a shielding device covers the first and second connectors to block radio waves. An opening is formed in the shielding device, and disposed between the first and second near field communication interfaces opposed to one another while the first and second connectors are in the connected state.

Preferably, at least one of the first and second connectors include a shielding cover disposed movably relative to the opening between open and closed positions, for closing the opening aligned with the first and second near field communication interfaces upon being set in the closed position, and for opening the opening upon being set in the open position.

Preferably, the first connector includes a plug unit, disposed to project toward the processing apparatus, for containing the first near field communication interface. The second connector includes a receiving cavity for engagement with the plug unit to set the first and second connectors in the connected state. The second near field communication interface is disposed on an inner wall of the receiving cavity, and the shielding device covers a surface of the second connector disposed around the receiving cavity.

Preferably, the second near field communication interface receives interface information of the first near field communication interface. Furthermore, an information acquisition device acquires authorization information of the first near field communication interface through a path different from the second near field communication interface upon setting the first and second connectors in the connected state. An authorizer authorizes the near field wireless communication between the first and second near field communication interfaces in case the interface information corresponds to the authorization information.

Preferably, furthermore, a memory is incorporated in the endoscope, for storing the authorization information. The information acquisition device is incorporated in the processing apparatus, and accesses the memory upon setting the first and second connectors in the connected state.

Preferably, there are at least first and second communication paths, which are provided in parallel with one another between the first and second connectors, and each of which includes the first and second near field communication interfaces, for simultaneously performing the near field wireless communication upon setting of the first and second connectors in the connected state.

Preferably, the second near field communication interface is so disposed that transmission of radio waves is blocked by a distance between the first and second communication paths.

Preferably, furthermore, a separating wall is disposed with the first near field communication interface, for blocking radio waves between the first and second communication paths.

Preferably, the second near field communication interface is enabled one after another in relation to the first and second communication paths upon setting of the first and second connectors in the connected state.

Preferably, the first near field communication interface is enabled one after another in relation to the first and second communication paths upon setting of the first and second connectors in the connected state.

Preferably, the endoscope includes a data division unit for dividing data of the image signal from the imaging unit into divided packet data, and transmitting the divided packet data to the first and second communication paths in distribution. The processing apparatus includes a data restoration unit for receiving the divided packet data from the first and second communication paths, and restoring the image signal.

Preferably, in relation to each of the first and second communication paths, the second near field communication interface receives interface information of the first near field communication interface. Furthermore, an information acquisition device acquires authorization information of the first near field communication interface through a path different from the second near field communication interface upon setting the first and second connectors in the connected state. An authorizer authorizes the near field wireless communication between the first and second near field communication interfaces in case the interface information corresponds to the authorization information in relation to both of the first and second communication paths.

Preferably, furthermore, a light source apparatus is connected with the endoscope, for generating light to illuminate the object. The first and second connectors function also for connection to the light source apparatus.

Consequently, electric noise can be prevented reliably in the use of ratio communication with the processing apparatus, because a connected state between connectors is monitored for the purpose of starting the NFC communication between near field communication interfaces.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an endoscope system;

FIG. 2 is a block diagram schematically illustrating electric circuits in the endoscope system;

FIG. 3 is a perspective view illustrating a communication connector;

FIG. 4 is a vertical section illustrating the communication connector and a receptacle connector;

FIG. 5 is a perspective view illustrating the receptacle connector;

FIG. 6 is a vertical section illustrating a connected state of the communication connector and the receptacle connector;

FIG. 7 is a perspective view illustrating another preferred communication connector with a shielding cover;

FIG. 8 is a vertical section illustrating the communication connector and a receptacle connector;

FIG. 9 is an explanatory view illustrating the receptacle connector;

FIG. 10 is a vertical section illustrating a connected state of the communication connector and the receptacle connector;

FIG. 11 is a block diagram schematically illustrating a second preferred endoscope system;

FIG. 12 is a perspective view illustrating a communication connector;

FIG. 13 is a vertical section illustrating the communication connector and a receptacle connector;

FIG. 14 is a cross section illustrating the communication connector;

FIG. 15 is a perspective view illustrating the receptacle connector;

FIG. 16 is a vertical section illustrating a connected state of the communication connector and the receptacle connector;

FIG. 17 is a block diagram schematically illustrating a third preferred endoscope system;

FIG. 18 is a perspective view illustrating a communication connector;

FIG. 19 is a vertical section illustrating the communication connector and a receptacle connector;

FIG. 20 is a block diagram schematically illustrating a fourth preferred endoscope system.

### DETAILED DESCRIPTION OF THE PREFERRED EMBODIMENT(S) OF THE PRESENT INVENTION

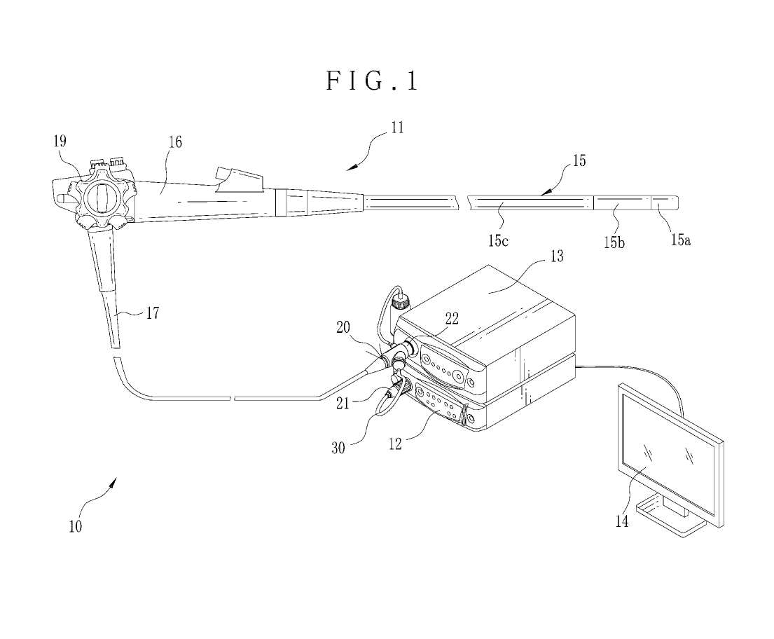

In FIG. 1, an endoscope system 10 includes an electronic endoscope 11, a processing apparatus 12, a light source apparatus 13 and a monitor display panel 14. The endoscope 11 images an object in a body cavity of a human body. The processing apparatus 12 forms an endoscopic image according to an image signal from the endoscope 11. The light source apparatus 13 generates light for lighting the object. The display panel 14 displays the endoscopic image. The endoscope 11 includes an elongated tube 15 or insertion tube, a grip handle 16 and a universal cable 17. The elongated tube 15 is entered in the body. The grip handle 16 is disposed at a proximal end of the elongated tube 15. The universal cable 17 extends for connection of the grip handle 16 to the processing apparatus 12 and the light source apparatus 13.

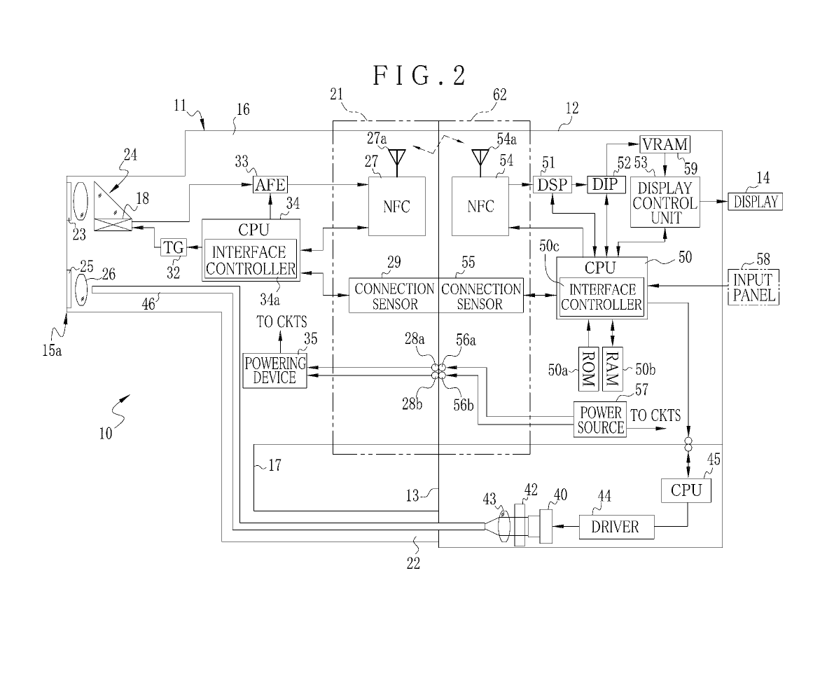

The elongated tube 15 includes a tip device 15a, a steering device 15b and a flexible device 15c. The tip device 15a is a rigid structure and contains a CCD image sensor 18. See FIG. 2. The steering device 15b is disposed at a proximal end of the tip device 15a and bendable up and down and to the right and left. The flexible device 15c is disposed between the steering device 15b and the grip handle 16. Steering wheels 19 are disposed on the grip handle 16, and rotated to bend the steering device 15b in the various directions. The tip device 15a can be directed as required in a body cavity by manipulating the steering device 15b.

A composite connector 20 is provided at a proximal end of the universal cable 17 on the side of the processing apparatus 12 and the light source apparatus 13. The composite connector 20 includes a communication connector 21 (first connector), a light source connector 22 and a branch cable 30, and connects the endoscope 11 to the processing apparatus 12 and the light source apparatus 13 in a removable manner.

The processing apparatus 12 electrically connected to the light source apparatus 13 controls various elements of the endoscope system 10. Near field communication interfaces 27 and 54 or NFC interfaces (first and second interfaces) and a signal cables are utilized in the processing apparatus 12 to receive an image signal output by the CCD image sensor 18. The processing apparatus 12 creates image data by image processing of the image signal. The display panel 14 is driven by the processing apparatus 12 to display an endoscopic image according to the image data from the processing apparatus 12. A transmission cable is extended through the elongated tube 15, the grip handle 16 and a tube of the universal cable 17. The processing apparatus 12 supplies the endoscope 11 with power by means of contact terminals 28a, 28b, 56a and 56b and the transmission cable.

In FIG. 2, the tip device 15a includes a viewing window 23, a lens system 24, the CCD image sensor 18, lighting windows 25 and lighting lenses 26. The communication connector 21 has the near field communication interface 27 and the contact terminals 28a and 28b, and also is provided with a connection detection device 29 (connection sensor). The grip handle 16 has a timing generator 32 (TG), an analog signal processor 33 (AFE), a CPU 34 and a powering device 35.

The lens system 24 includes a lens, prism and other optical elements, and focuses light from an object in the body cavity to the CCD image sensor 18 upon passage through the viewing window 23. The lighting windows 25 apply light to the object in the body cavity. The light is supplied by the light source apparatus 13 to the endoscope 11. A light guide device 46 is extended through the light source connector 22, the universal cable 17 and the elongated tube 15, and guides the light, which is passed through the lighting lenses 26 and applied to the object through the lighting windows 25.

The CCD image sensor 18 photoelectrically converts a focused image of the body cavity by use of the lens system 24. The CCD image sensor 18 has a plurality of pixels for outputting an image signal as pixel values according to a light amount of incident light. An image signal output by the CCD image sensor 18 is an analog signal, is processed by the analog signal processor 33 for noise reduction, gain correction and the like, is converted for A/D conversion, and is sent to the near field communication interface 27.

An antenna 27a is included in the near field communication interface 27, which also has a modulator/demodulator for modulation and demodulation to radio frequency, and a transmission/reception device for transmitting/receiving a radio signal. The near field communication interface 27 modulates the image signal from the analog signal processor 33, converts this into the radio signal of NFC communication (near field wireless communication), causes the antenna 27a to emit radio waves of the radio signal, and then receives radio waves from the processing apparatus 12 by use of the antenna 27a, demodulates those into a control signal, and inputs this to the CPU 34.

The timing generator 32 supplies the CCD image sensor 18 with a clock signal. The CCD image sensor 18 forms an endoscopic image in response to the clock signal, and outputs the image signal. To this end, the CPU 34 receives a control signal from a CPU 50 in the processing apparatus 12. In case the endoscope 11 communicates with the processing apparatus 12 through the near field communication interfaces 27 and 54 to be described later, the CPU 34 drives the timing generator 32 according to the control signal.

The analog signal processor 33 includes a correlated double sampling circuit (CDS), an automatic gain control device (AGC) and an A/D converter. The correlated double sampling circuit processes the image signal from the CCD image sensor 18 in the correlated double sampling, and eliminates electric noise generated by driving the CCD image sensor 18. The automatic gain control device amplifies the image signal after elimination of the noise. The A/D converter converts the image signal from the automatic gain control device into a digital image signal with a predetermined number of bits. The analog signal processor 33 is controlled by the CPU 34. The CPU 34 adjusts a gain for the image signal with the automatic gain control device according to a control signal from the CPU 50 in the processing apparatus 12.

An interface controller 34a is incorporated in the CPU 34. In case a detection signal is input by the connection detection device 29, the interface controller 34a enables the near field communication interface 27 for communicability in the NFC communication. The detection signal is information of a connected state of the communication connector 21 of the endoscope 11 with a receptacle connector 62 (second connector) or socket device of the processing apparatus 12. The powering device 35, upon supply of power from the power source in the processing apparatus 12 through the contact terminals 28a and 28b and the contact terminals 56a and 56b, starts powering the elements in the endoscope 11.

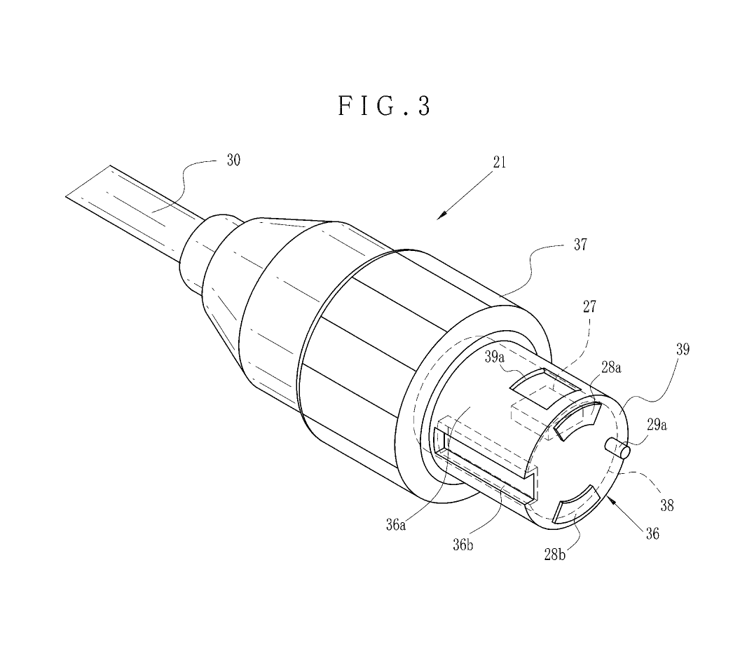

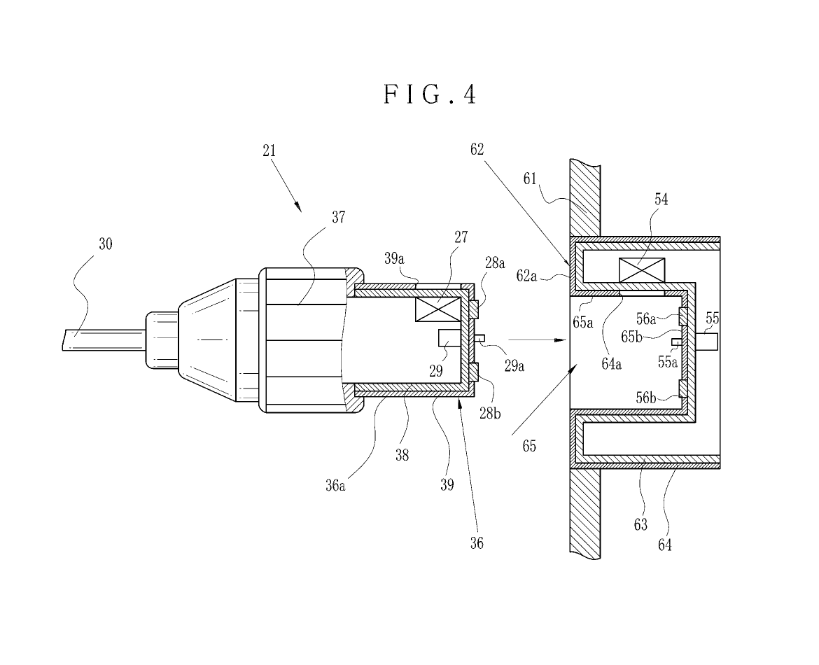

In FIGS. 3 and 4, the communication connector 21 is provided at a proximal end of the branch cable 30, and includes a plug unit 36 or plug head for engagement, and a finger grip 37. The branch cable 30 is a branch from the universal cable 17 extending inside the light source connector 22, and protrudes from an outer surface of the light source connector 22 to extend to the communication connector 21. In FIG. 4, partial elements are not depicted, such as lines for connecting the contact terminals 28a and 28b to the powering device 35, and lines for connecting a power source 57 with the contact terminals 56a and 56b.

The plug unit 36 includes a connector housing 38 and a shielding device 39 (shield wall). The connector housing 38 is cylindrical and has a closed proximal end directed to the processing apparatus 12. The shielding device 39 covers a surface of the connector housing 38. The plug unit 36 is in a shape projecting at the proximal end from the finger grip 37 toward the processing apparatus 12. The plug unit 36 has a cylindrical outer surface 36a and a key way 36b, which is formed in the outer surface 36a partially from the proximal end. A receiving cavity 65 is formed in the receptacle connector 62 as will be described later, and becomes engaged with the outer surface 36a. A key projection 65c is disposed in the receiving cavity 65, and becomes engaged with the key way 36b.

The near field communication interface 27 is contained in the connector housing 38. The connector housing 38 is formed from a radio-transmissive material, for example, plastic material. The near field communication interface 27 is disposed to contact an inner wall of the connector housing 38 and in an inner space under the outer surface 36a.

The shielding device 39 covers an outer surface and proximal end of the connector housing 38. An opening 39a is formed in the connector housing 38 to extend in its axial direction. The opening 39a has a portion aligned with the near field communication interface 27 in the axial direction and the circumferential direction of the plug unit 36. The shielding device 39 is formed from electrically conductive material, and blocks radio wave, for example, aluminum and other metals. Note that the shielding device 39 can be formed by metal plating of the surface of the connector housing 38, or can be initially prepared by sheet metal working separately from the connector housing 38 and then attached to the connector housing 38.

Only a component of the radio waves passed through the opening 39a can be transmitted externally in the radio waves originally emitted by the near field communication interface 27. Only a component of the radio waves passed through the opening 39a can be received by the near field communication interface 27 in the radio waves entered from the outside.

The finger grip 37 is between a distal end of the connector housing 38 and a proximal end of the branch cable 30. A peripheral surface of the finger grip 37 has a corrugation or projections and notches in a form of a dial. The contact terminals 28a and 28b are provided at a proximal end of the plug unit 36, and protrude on the proximal side of the plug unit 36. It is possible with the contact terminals 28a and 28b to carry out reliable connection to the contact terminals 56a and 56b of the processing apparatus 12. Furthermore, the contact terminals 28a and 28b are positioned in a rotationally symmetric manner with 180 degrees about the center of the plug unit 36. Even in case the plug unit 36 is inclined with reference to the receiving cavity 65 of the receptacle connector 62 and engaged therewith, at least one of the contact terminals 28a and 28b can be reliably connected. Note that an insulator of a non-conductive material is fitted around the contact terminals 28a and 28b for preventing conduction with the shielding device 39.

The connection detection device 29 is incorporated in the plug unit 36. The connection detection device 29 is a mechanical switch, and in case a pin projection 29a or button is depressed, is turned on, and in case the pin projection 29a is released from pressure, is turned off and becomes protruded from the plug unit 36. Turn-on of the connection detection device 29 generates a detection signal of a connected state between the communication connector 21 of the endoscope 11 and the receptacle connector 62 of the processing apparatus 12 to input this to the CPU 34.

The light source apparatus 13 includes a light source 40, a wavelength filter 42 for wavelength conversion, a condensing lens 43, a light source driver 44 and a CPU 45. An example of the light source 40 is a light-emitting diode (LED). The wavelength filter 42 converts light from the light source 40 into normal white light. The light from the light source 40 is condensed by the condensing lens 43 and guided to an entrance end of the light guide device 46.

The light guide device 46 is a bundle constituted by plural optical fibers of quartz and winding tape for bundling the optical fibers. Light conducted to an exit end of the light guide device 46 is diffused by the lighting lenses 26, and applied to an object in a body cavity.

The light source driver 44 adjusts a light amount of the light emitted by the light source 40 by changing the voltage applied to the light source 40. The CPU 45 communicates with the CPU 50 in the processing apparatus 12, and controls the light source driver 44.

The processing apparatus 12 includes the CPU 50, the near field communication interface 54, the contact terminals 56a and 56b and the power source 57, and also a digital signal processor 51 (DSP), a digital image processor 52 (DIP), a display control unit 53, a connection detection device 55 (connection sensor) and an input panel 58 or user interface.

The CPU 50 is connected to various elements by a data bus, address bus and control lines (all not shown), and controls the elements in the processing apparatus 12. A ROM 50a stores various programs (operation system (OS), application programs and the like) and various data (graphic data and the like) for controlling the processing apparatus 12. The CPU 50 reads programs and data from the ROM 50a as required. A RAM 50b is a working memory with which the CPU 50 processes the programs and data sequentially. Also, the CPU 50 acquires information from the input panel 58, the LAN (local area network) and the like, and writes the information to the RAM 50b, the information including attribute information such as a date, subject body, physician and the like of diagnosis in a form of alphanumeric information.

The digital signal processor 51 processes the image signal generated from the CCD image sensor 18 and input by the near field communication interfaces 27 and 54 for various functions of the image processing, such as color separation, color interpolation, gain correction, white balance adjustment, gamma correction and the like, and generates image data. The image data from the digital signal processor 51 is input to a working memory in the digital image processor 52.

The digital image processor 52 processes the image data from the digital signal processor 51 in the image processing of various functions. There is a VRAM 59 to which the processed image data from the digital image processor 52 is stored temporarily. Then the image data is input to the display control unit 53.

The VRAM 59 is a memory for temporarily storing endoscopic images from the digital image processor 52, and stores plural images, for example, five images at the same time. Assuming that a new endoscopic image is input in a state of storing images of the maximum number (5) in the VRAM 59, then an oldest one of the images is abandoned to write the image to the VRAM 59 in an overwritten manner. In short, the VRAM 59 stores a plurality of newest images, or 5 newest images, according to the storage capacity. The display control unit 53 selectively reads an image of one frame among the images of the plural frames in the VRAM 59, and drives the display panel 14 to display the image.

The display control unit 53 selectively reads an endoscopic image from the VRAM 59, and drives the display panel 14 to display the endoscopic image. According to an image selection signal from the CPU 50, the display control unit 53 reads an endoscopic image of one frame among plural endoscopic images stored in the VRAM 59, and causes the display panel 14 to display the endoscopic image.

The near field communication interface 54 is disposed in the receptacle connector 62, and constructed according to the standards of the NFC communication of the near field communication interface 27. An antenna 54a is included in the near field communication interface 54 together with a modulator/demodulator and a transmission/reception device. The near field communication interface 54 modulates a control signal from the CPU 50, converts this into a radio signal, causes the antenna 54a to emit radio waves of the radio signal, and then receives radio waves from the near field communication interface 27 by use of the antenna 54a, demodulates those into an image signal, and inputs this to the digital signal processor 51.

The power source 57 is connected to a commercial power source, and supplies the elements with power in the processing apparatus 12. While the contact terminals 56a and 56b are connected with the contact terminals 28a and 28b, the power source 57 supplies the powering device 35 in the endoscope 11 with power through the contact terminals.

An interface controller 50c is incorporated in the CPU 50. In case a detection signal is input by the connection detection device 55, the interface controller 50c enables the near field communication interface 54 for communicability in the NFC communication. The detection signal is information of a connected state of the communication connector 21 of the endoscope 11 with the receptacle connector 62 of the processing apparatus 12.

An apparatus housing 61 is included in the processing apparatus 12. The input panel 58 is disposed on the apparatus housing 61. Examples of the input panel 58 are a touchscreen, mouse, keyboard, and other input devices. The CPU 50 controls various elements in the endoscope system 10 according to an input signal from the input panel 58.

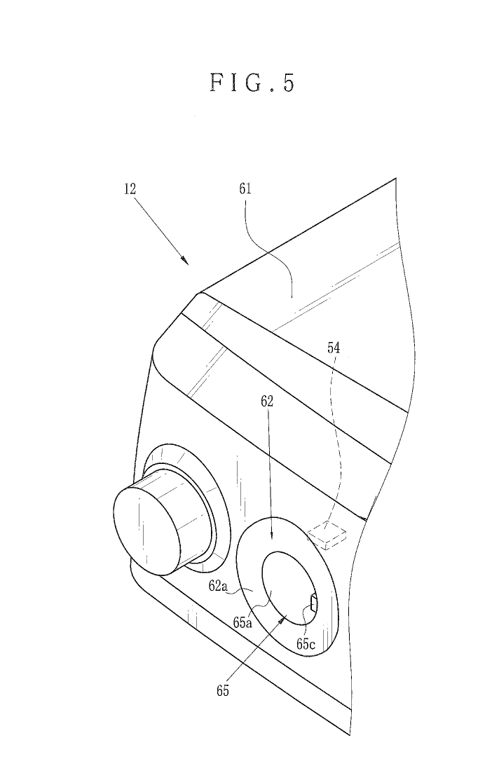

In FIGS. 4 and 5, the receptacle connector 62 is disposed on the apparatus housing 61 of the processing apparatus 12. The receptacle connector 62 includes a socket housing 63 and a shielding device 64 (shield wall) for covering a surface of the socket housing 63. The receptacle connector 62 has a cylindrical form, and has a front end 62a. The receiving cavity 65 is formed at the center to extend to the inside from the front end 62a.

The receiving cavity 65 is cylindrical and concentric with an outer surface of the receptacle connector 62, and includes an inner surface 65a, a flat receiving surface 65b and the key projection 65c. The flat receiving surface 65b is parallel with the front end 62a of the receptacle connector 62. The key projection 65c projects from the inner surface 65a.

The near field communication interface 54 is incorporated in the socket housing 63. The socket housing 63 is formed from a radio-transmissive material, for example, plastic material. The near field communication interface 54 is disposed to contact an inner wall of the socket housing 63 and in an inner space under the inner surface 65a. A relative position of the near field communication interface 54 and the key projection 65c corresponds to a relative position of the near field communication interface 27 and the key way 36b. Engagement of the key way 36b with the key projection 65c positions the plug unit 36 and the receiving cavity 65 in the circumferential direction and the axial direction exactly to align the near field communication interface 27 with the near field communication interface 54.

The shielding device 64 covers an outer surface and distal end of the socket housing 63, and also extends to the inner surface 65a of the receiving cavity 65. An opening 64a is formed in the shielding device 64 and disposed in the inner surface 65a of the receiving cavity 65. The opening 64a is aligned with the near field communication interface 54 in the axial direction and circumferential direction of the receiving cavity 65. An inner diameter of the inner surface 65a is determined according to the outer surface 36a of the communication connector 21, to receive the outer surface 36a on the inner surface 65a.

The shielding device 64 is a material formed from the same material as the shielding device 39 of the communication connector 21, and blocks radio waves. Only a component of the radio waves passed through the opening 64a can be transmitted externally in the radio waves originally emitted by the near field communication interface 54. Only a component of the radio waves passed through the opening 64a can be received by the near field communication interface 54 in the radio waves entered from the outside.

An example of the near field communication interfaces 27 and 54 is an interface device according to the standards of TransferJet (trade name) for the NFC communication. Note that it is possible to use other interface devices for the near field communication interfaces 27 and 54.

The NFC communication is radio communication in which interfaces are communicable to each other only in case those are located near to each other. In the present embodiment, the near field communication interfaces 27 and 54 are communicable in case a distance between those is as short as 1-3 cm for receiving radio waves. The near field communication interfaces 27 and 54 automatically start the NFC communication in case those are enabled and also located near to one another.

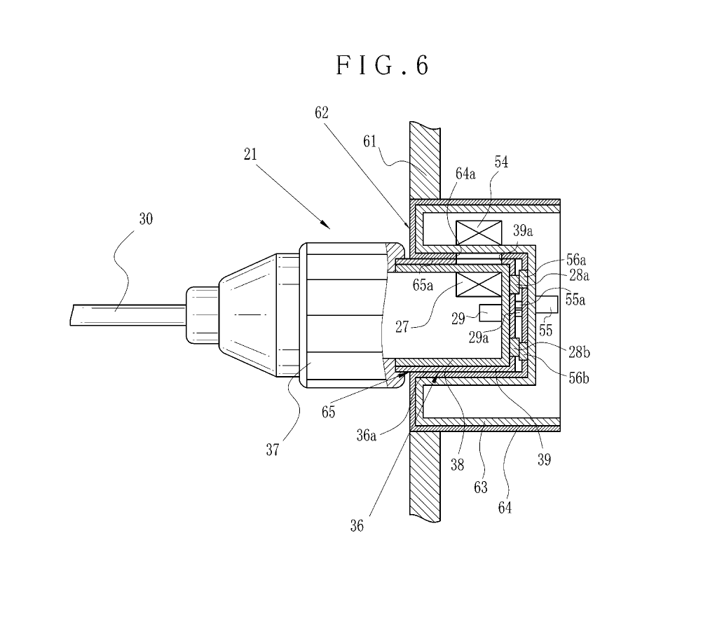

In FIG. 6, the key way 36b and the key projection 65c position the communication connector 21 and the receptacle connector 62 in the circumferential direction and the axial direction. The outer surface 36a of the plug unit 36 is engaged with the inner surface 65a of the receiving cavity 65 to couple the communication connector 21 to the receptacle connector 62 tightly without drop, so as to oppose the near field communication interfaces 27 and 54 to one another. The near field communication interfaces 27 and 54 are communicable in the NFC communication because a distance between those is as short as 1-3 cm. While the NFC communication is established, the near field communication interfaces 27 and 54 transmit an image signal from the endoscope 11 to the processing apparatus 12 and transmit a control signal from the processing apparatus 12 to the endoscope 11.

The openings 39a and 64a are formed in the shielding devices 39 and 64 of the communication connector 21 and the receptacle connector 62 and aligned with the near field communication interfaces 27 and 54. While the communication connector 21 is coupled to the receptacle connector 62 tightly without drop, the openings 39a and 64a are so disposed as to oppose the near field communication interface 27 to the near field communication interface 54. Thus, components of radio waves different from those passing the openings 39a and 64a are blocked by the shielding devices 39 and 64 while the NFC communication is established between the near field communication interfaces 27 and 54.

The contact terminals 56a and 56b are disposed on the flat receiving surface 65b of the receiving cavity 65, and opposed to the contact terminals 28a and 28b incorporated in the connector housing 38. In case the connector housing 38 is mounted in the socket housing 63 to couple the communication connector 21 to the receptacle connector 62 tightly without drop, then the contact terminals 56a and 56b become connected with respectively the contact terminals 28a and 28b. A peripheral surface of the contact terminals 56a and 56b is covered with non-conductive material for preventing conduction with the shielding device 64.

The connection detection device 55 is disposed in a space under the flat receiving surface 65b. The connection detection device 55 is a mechanical switch similar to the connection detection device 29, and in case a pin projection 55a or button is depressed, is turned on, and in case the pin projection 55a is released from pressure, is turned off and becomes protruded from the flat receiving surface 65b. Turn-on of the connection detection device 55 generates a detection signal of a connected state between the communication connector 21 of the endoscope 11 and the receptacle connector 62 of the processing apparatus 12 to input this to the CPU 34.

The operation of the endoscope system 10 is described now. For diagnosis, at first the composite connector 20 of the endoscope 11 is connected to the processing apparatus 12 and the light source apparatus 13. To couple the communication connector 21 of the composite connector 20 with the receptacle connector 62 of the processing apparatus 12, the key projection 65c is aligned with the key way 36b. The outer surface 36a of the plug unit 36 is engaged with the inner surface 65a of the receiving cavity 65, to couple the communication connector 21 with the receptacle connector 62 tightly without drop. Then the near field communication interface 27 is set close to the near field communication interface 54. The contact terminals 28a and 56a contact respectively the contact terminals 28b and 56b. The power supply for the processing apparatus 12 is turned on, to supply the elements in the processing apparatus 12 with power. The powering device 35 of the endoscope 11 is also supplied with power by the contact terminals 28a, 28b, 56a and 56b. The powering device 35 supplies the elements in the endoscope 11 with power.

In case the power supply for the processing apparatus 12 is turned on and the communication connector 21 becomes coupled with the receptacle connector 62, then the connection detection devices 29 and 55 are turned on, to input a detection signal to the CPUs 34 and 50. The CPU 34 enables the near field communication interface 27. The CPU 50 enables the near field communication interface 54. In response, the near field communication interfaces 27 and 54 start the NFC communication (near field wireless communication) because located close to one another.

The diagnosis is ready. The tip device 15a of the endoscope 11 is entered in a gastrointestinal tract, for example, large intestine of a patient. Light from the light source apparatus 13 is passed through the light guide device 46, the lighting lenses 26 and the lighting windows 25 in the tip device 15a, and applied to an intestinal wall of the large intestine. A control signal is transmitted from the processing apparatus 12 to the endoscope 11 through the near field communication interfaces 27 and 54, so that the CCD image sensor 18 in the tip device 15a is driven. The CCD image sensor 18 images an object of the intestinal wall and outputs an image signal. The near field communication interfaces 27 and 54 transmit the image signal to the processing apparatus 12, which processes the image signal for the image processing, and drives the display panel 14 to display an endoscopic image of the image signal. A physician or user views the intestinal wall with the display panel 14.

As described heretofore, the NFC communication is established between the near field communication interfaces 27 and 54 located near to one another upon coupling the communication connector 21 to the receptacle connector 62. Thus, the communication between the endoscope 11 and the processing apparatus 12 can be reliable. The CCD image sensor 18 can function stably to form an endoscopic image. As the NFC communication is established upon enabling the near field communication interfaces 27 and 54 according to detecting the connected state between the communication connector 21 and the receptacle connector 62, it is possible during the NFC communication to prevent other interface devices from coming near to the near field communication interfaces 27 and 54 contained in the communication connector 21 and the receptacle connector 62. It is unnecessary to emit radio waves to a wide area or long distance owing to the NFC communication. Thus, it is possible to prevent a problem of electric noise due to crosstalk of radio waves, and keep security in the transmission.

Owing to the NFC communication with the near field communication interfaces 27 and 54, it is possible to reduce laborious operation of a physician or user, as no complicated setting of the wireless LAN standards or the like is required. No shielding structure for a large area of the endoscope 11 and the processing apparatus 12 is required in consideration of preventing a problem of electric noise of crosstalk and keeping security in the transmission, so that a manufacturing cost can be reduced. Also, the surfaces of the connector housing 38 and the socket housing 63 are covered by the shielding devices 39 and 64 for containing the near field communication interfaces 27 and 54, to block external radio waves. Thus, it is possible to prevent a problem of electric noise due to crosstalk, and keep security in the transmission.

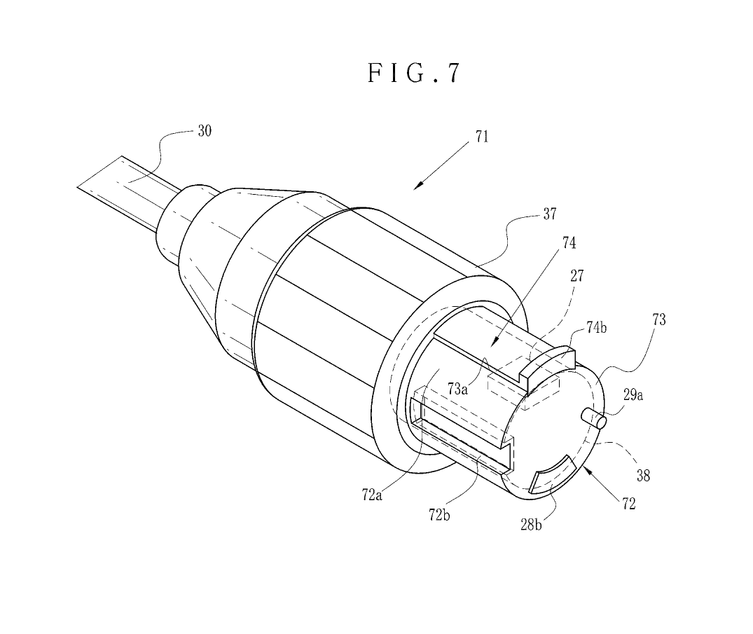

In the first embodiment, the shielding devices 39 and 64 are the walls. In FIGS. 7-10, another preferred structure is illustrated, in which a shielding device is partially slidable, and while not used, is in a closed position for blocking radio waves, and while in a connected state, is slid to an open position from the closed position. Elements similar to those of the first embodiment are designated with identical reference numerals. In FIGS. 7-10, the contact terminals 28a and 56a are not illustrated for the simplicity in the depiction. However, those can be arranged in a similar manner to the first embodiment. Also, only the contact terminals 28b and 56b can be used without others as illustrated in FIGS. 7-10.

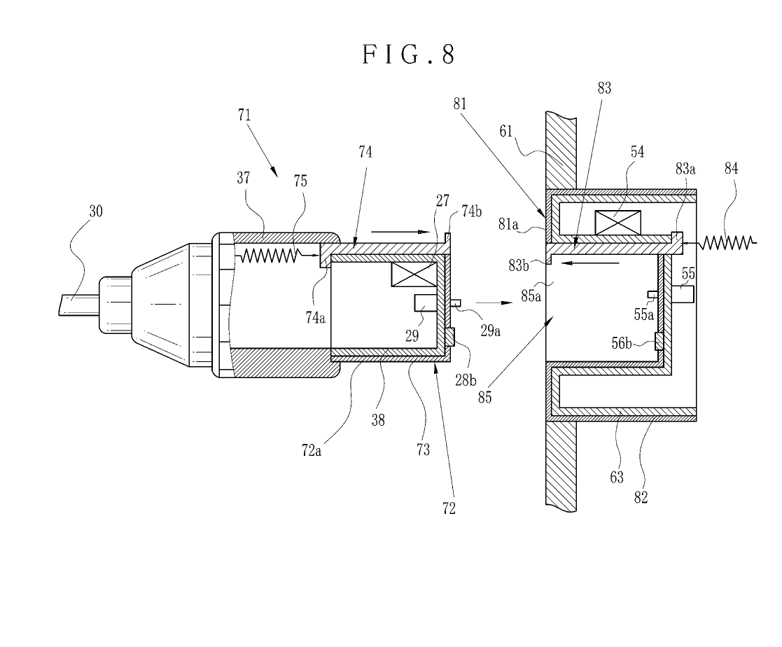

In FIGS. 7 and 8, another preferred communication connector 71 (first connector) of the example includes a plug unit 72 or plug head for engagement, and the finger grip 37. The plug unit 72 includes a shielding device 73 (shield wall), a shielding cover 74 and a compression coil spring 75. The shielding device 73 covers the connector housing 38. The plug unit 72 includes an outer surface 72a and a key way 72b formed in the outer surface 72a in a manner similar to the communication connector 21 described above.

The shielding device 73 covers an outer surface and a proximal end of the connector housing 38. An opening 73a is formed in the connector housing 38 to extend in its axial direction. The opening 73a has a portion aligned with the near field communication interface 27 in the axial direction and the circumferential direction with the connector housing 38. The shielding cover 74 is as wide as the opening 73a, and received therein in a slidable manner. An anti-drop projection 74a is formed at a distal end of the shielding cover 74. A target projection 74b is formed at a proximal end of the shielding cover 74. The shielding cover 74 is movable between open and closed positions, and upon being set in the closed position, covers a portion of the opening 73a aligned with the near field communication interface 27 in the axial direction and blocks radio waves, and upon being set in the open position, is shifted away from the closed position. The anti-drop projection 74a is fitted on a distal end of the connector housing 38, and prevents the shielding cover 74 in the closed position from dropping away toward the proximal end of the connector housing 38.

The compression coil spring 75 is disposed between the anti-drop projection 74a and the finger grip 37, and biases the shielding cover 74 toward the closed position. A plate spring or the like can be used in place of the compression coil spring 75 for biasing the shielding cover 74. A thickness of the shielding cover 74 is greater than the shielding device 73, and projects externally from the shielding device 73. The target projection 74b further protrudes externally. Entry of the communication connector 71 into a receiving cavity 85 of a receptacle connector 81 (second connector) or socket device causes a front end 81a of the receptacle connector 81 to press the target projection 74b, which moves from the closed position to the open position against the compression coil spring 75. While the shielding cover 74 is in the open position, passage of radio waves through the opening 73a is allowed for transmission and reception with the near field communication interface 27.

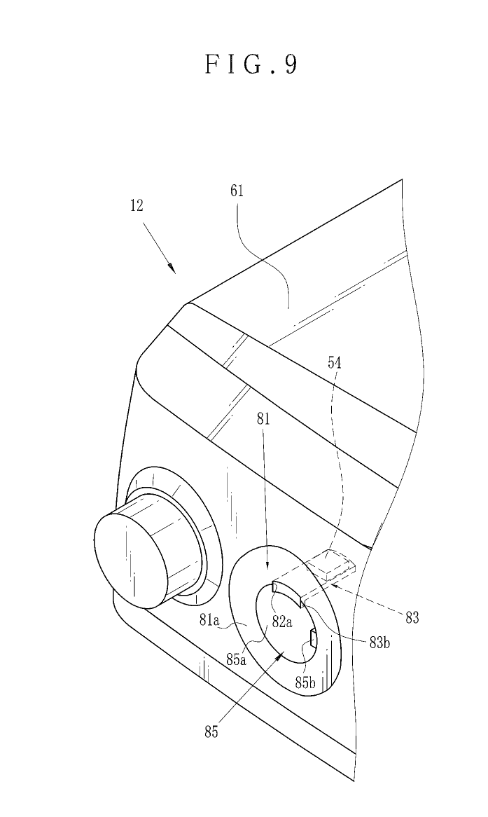

In FIGS. 8 and 9, the receptacle connector 81 of the embodiment includes a shielding device 82 (shield wall), a shielding cover 83 and a compression coil spring 84 with the socket housing 63. In a manner similar to the receptacle connector 62, the receptacle connector 81 has a stepped shape with the front end 81a. The receptacle connector 81 includes the receiving cavity 85 and a key projection 85b. The receiving cavity 85 is engaged with the plug unit 72 of the communication connector 71. The key projection 85b is formed on an inner surface 85a of the receiving cavity 85. The shielding device 82 covers an outer surface and a distal end of the socket housing 63, and extends to the surface of the receiving cavity 85.

A relative position of the near field communication interface 54 and the key projection 85b corresponds to a relative position of the near field communication interface 27 and the key way 72b in a manner similar to the first embodiment. Engagement of the key way 72b with the key projection 85b positions the plug unit 72 and the receiving cavity 85 in the circumferential direction and the axial direction, to position the near field communication interface 27 at the near field communication interface 54.

An opening 82a is formed with the receiving cavity 85 to extend in its axial direction. The opening 82a has a portion aligned with the near field communication interface 54 in the axial direction and the circumferential direction in an inner surface of the receiving cavity 85. The shielding cover 83 is as wide as the opening 82a, and received therein in a slidable manner. An anti-drop projection 83a is formed at a proximal end of the shielding cover 83. A target projection 83b is formed at a distal end of the shielding cover 83. The shielding cover 83 is movable between open and closed positions, and upon being set in the closed position, covers a portion of the receiving cavity 85 aligned with the near field communication interface 54 in the axial direction, and upon being set in the open position, is shifted away from the closed position. The anti-drop projection 83a is fitted on an inner wall of the socket housing 63, and prevents the shielding cover 83 in the closed position from moving away toward the distal end of the socket housing 63.

The compression coil spring 84 is connected between a support portion (not shown) in the apparatus housing 61 and the anti-drop projection 83a, and biases the shielding cover 83 toward the closed position. A plate spring or the like can be used in place of the compression coil spring 84 for biasing the shielding cover 83. A thickness of the shielding cover 83 is greater than the shielding device 82, so that the shielding cover 83 projects into the receiving cavity 85 to decrease a radius from the shielding device 82. Entry of the plug unit 72 of the communication connector 71 into the receiving cavity 85 of the receptacle connector 81 causes the plug unit 72 to press the shielding cover 83, which moves from the closed position to the open position against the compression coil spring 84. While the shielding cover 83 is in the open position, passage of radio waves through the opening 82a is allowed for transmission and reception with the near field communication interface 54.

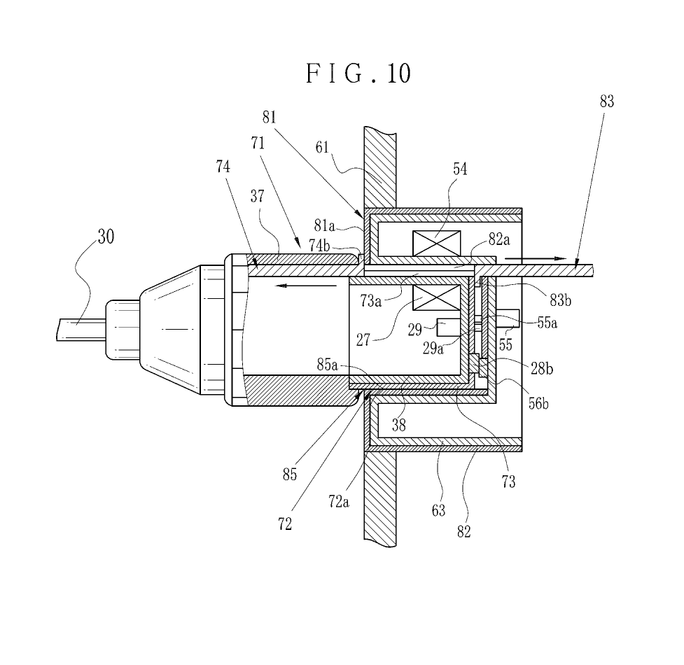

In FIG. 10, the key way 72b and the key projection 85b position the communication connector 71 and the receptacle connector 81 in the circumferential direction and the axial direction. The outer surface 72a of the plug unit 72 is engaged with the inner surface 85a of the receiving cavity 85 to couple the communication connector 71 to the receptacle connector 81 tightly without drop, so that a proximal end of the plug unit 72 and a distal end of the receptacle connector 81 push the shielding covers 74 and 83 to the open position. The openings 73a and 82a are so aligned together as to oppose the near field communication interfaces 27 and 54 to one another. While the NFC communication is established between the near field communication interfaces 27 and 54, components of radio waves different from that passed through the openings 73a and 82a are blocked by the shielding devices 73 and 82.

### Second Embodiment

In contrast with the near field communication interfaces 27 and 54 as one pair (communication path) in the first embodiment, a plurality of pairs (communication paths) of near field communication interfaces are provided in the second embodiment. Elements similar to those of the above embodiment are designated with identical reference numerals.

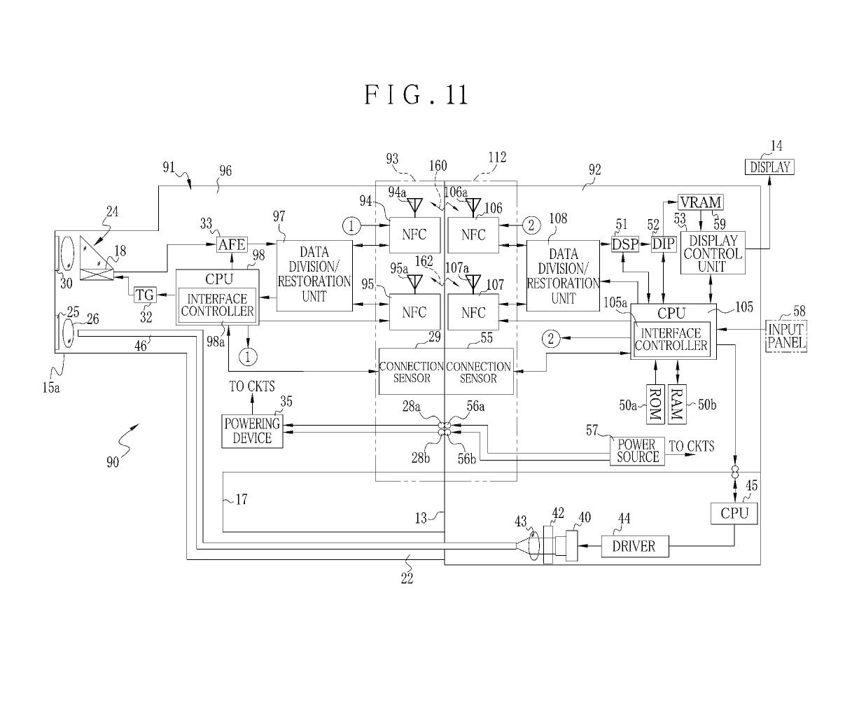

In FIG. 11, an endoscope system 90 includes an electronic endoscope 91 and a processing apparatus 92. A communication connector 93 (first connector) for the endoscope 91 includes near field communication interfaces 94 and 95 or NFC interfaces (first interfaces), the contact terminals 28a and 28b and the connection detection device 29. A grip handle 96 includes a data division/restoration unit 97, a CPU 98, the timing generator 32, the analog signal processor 33 and the powering device 35. The data division/restoration unit 97 divides an image signal of one frame into two divided packet data, sends those to the near field communication interfaces 94 and 95 in a distributed manner, and also restores a control signal from two divided packet data input by the near field communication interfaces 94 and 95, and inputs the control signal to the CPU 98.

The near field communication interfaces 94 and 95 modulate divided packet data divided by the data division/restoration unit 97 into a radio signal, which antennas 94a and 95a transmit by way of radio waves. Also, the radio waves emitted by the processing apparatus 92 are received by the antennas 94a and 95a, from which a radio signal is demodulated and input to the data division/restoration unit 97 as divided packet data.

An interface controller 98a is incorporated in the CPU 98. In case a detection signal is input by the connection detection device 29, the interface controller 98a enables the near field communication interfaces 94 and 95 successively for communicability in the NFC communication. The detection signal is information of a connected state of the communication connector 93 of the endoscope 91 with a receptacle connector 112 (second connector) or socket device of the processing apparatus 92. Note that a sequence of enabling the near field communication interfaces 94 and 95 with the interface controller 98a is predetermined. The near field communication interface 94 is enabled at first, and the near field communication interface 95 is enabled next.

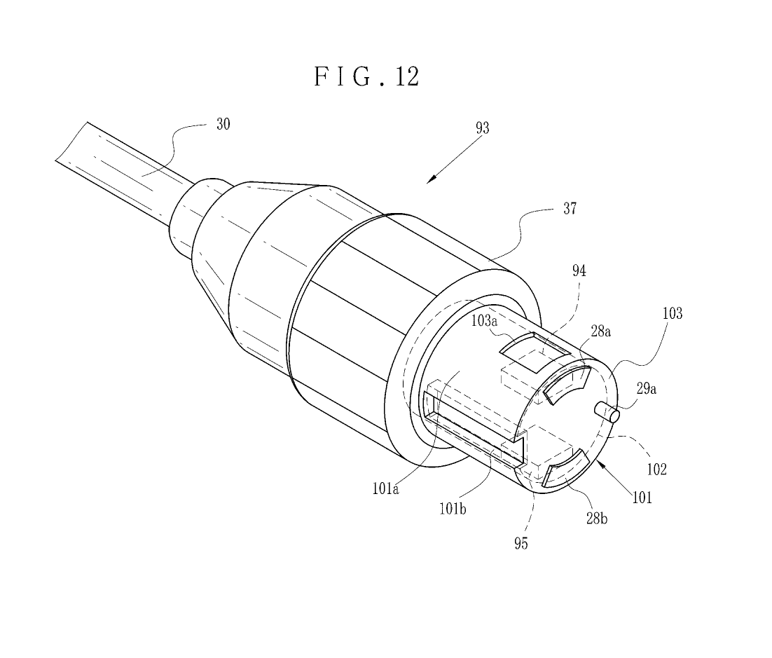

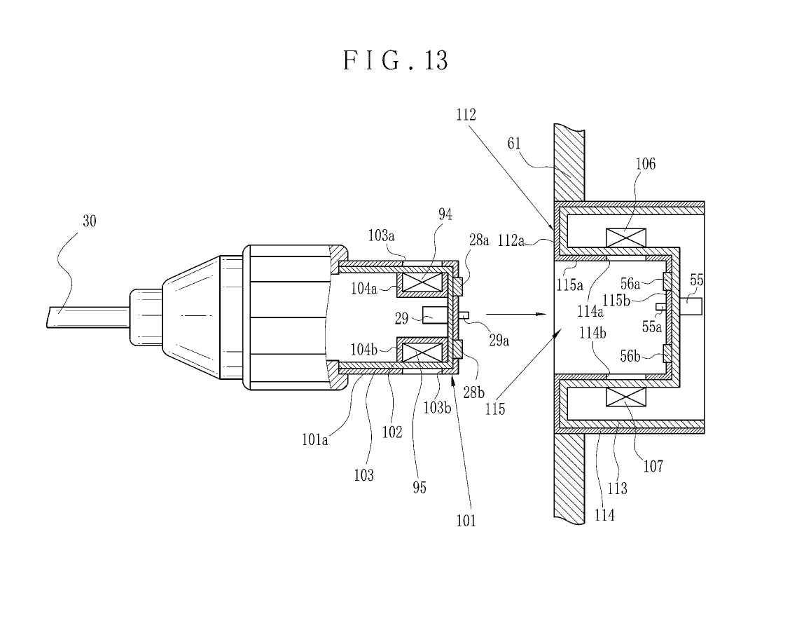

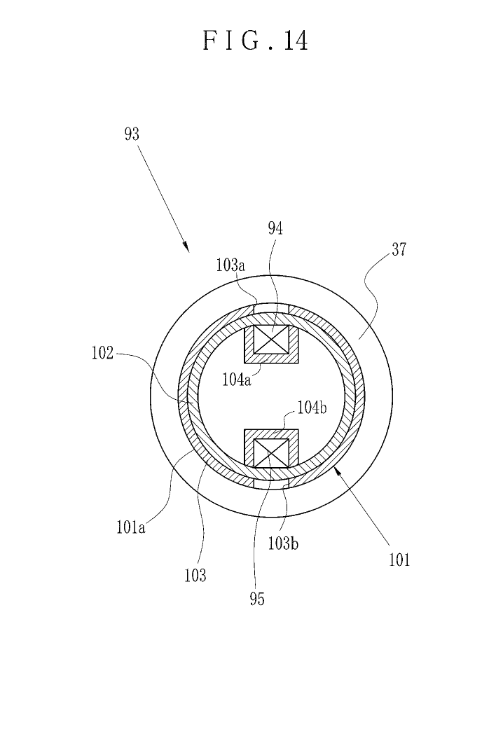

In FIGS. 12 and 13, the communication connector 93 is provided at a proximal end of the branch cable 30, and includes a plug unit 101 or plug head for engagement, and the finger grip 37. The plug unit 101 includes a connector housing 102, a shielding device 103 (shield wall) and separating walls 104a and 104b. See FIGS. 13 and 14. The connector housing 102 is cylindrical and has a closed proximal end opposed to the processing apparatus 92. The shielding device 103 covers a surface of the connector housing 102. The plug unit 101 is in a shape projecting toward the proximal end and toward the processing apparatus 92 from the finger grip 37. The plug unit 101 has a cylindrical outer surface 101a and a key way 101b, which is formed in the outer surface 101a partially from the proximal end. A receiving cavity 115 is formed in the receptacle connector 112 as will be described later, and becomes engaged with the outer surface 101a. A key projection 115c is disposed in the receiving cavity 115, and becomes engaged with the key way 101b.

The near field communication interfaces 94 and 95 are incorporated in the connector housing 102. The connector housing 102 is formed from a radio-transmissive material, for example, plastic material. The near field communication interfaces 94 and 95 are disposed to contact an inner wall of the connector housing 102 and in an inner space under the outer surface 101a. The near field communication interfaces 94 and 95 are disposed in a rotationally symmetric manner with 180 degrees about the center of the plug unit 101.

The shielding device 103 covers an outer surface and proximal end of the connector housing 102. Openings 103a and 103b are formed in the shielding device 103 to extend in its axial direction. The openings 103a and 103b have a portion aligned with the near field communication interfaces 94 and 95 in the axial direction and the circumferential direction with the plug unit 101. The shielding device 103 is formed from electrically conductive material, and blocks radio waves, in a manner similar to the shielding device 39 of the first embodiment.

The separating walls 104a and 104b block radio waves to be transmitted or received between the near field communication interfaces 94 and 95, and cover a large area around the near field communication interfaces 94 and 95 having a portion where the near field communication interfaces 94 and 95 are opposed to one another. In the embodiment, the separating walls 104a and 104b fully cover those except for their surface aligned with the openings 103a and 103b. The separating walls 104a and 104b are formed from electrically conductive material similar to the shielding devices 39 and 103, and block radio waves.

Only a component of the radio waves passed through the openings 103a and 103b can be transmitted externally in the radio waves originally emitted by the near field communication interfaces 94 and 95, in the structure with the shielding device 103 and the separating walls 104a and 104b. Only a component of the radio waves passed through the openings 103a and 103b can be received by the near field communication interfaces 94 and 95 in the radio waves entered from the outside. The near field communication interfaces 94 and 95, although aligned with one another, are prevented from communicating with one another by transmitting or receiving radio waves, because the separating walls 104a and 104b block radio waves emitted by the near field communication interfaces 94 and 95.

The processing apparatus 92 has a CPU 105, the digital signal processor 51, the digital image processor 52, the display control unit 53, the connection detection device 55, the contact terminals 56a and 56b, the power source 57 and the input panel 58. Also, the processing apparatus 92 includes near field communication interfaces 106 and 107 or NFC interfaces (second interfaces) and a data division/restoration unit 108.

The data division/restoration unit 108 divides one control signal from the CPU 105 into two divided packet data, which are distributed to the near field communication interfaces 106 and 107. The data division/restoration unit 108 restores an image signal according to two divided packet data input by the near field communication interfaces 106 and 107, and inputs the image signal to the digital signal processor 51.

The near field communication interfaces 106 and 107 are disposed in the receptacle connector 112 which will be described later, and constructed according to the standards of the NFC communication. The near field communication interfaces 106 and 107 modulate packet data divided by the data division/restoration unit 108 into a radio signal, which antennas 106a and 107a transmit by way of radio waves. Also, the radio waves emitted by the near field communication interfaces 94 and 95 of the endoscope 91 are received by the antennas 106a and 107a, from which a radio signal is demodulated and input to the data division/restoration unit 108 as divided packet data.

An interface controller 105a is incorporated in the CPU 105. In case a detection signal is input by the connection detection device 55, the interface controller 105a enables the near field communication interfaces 106 and 107 successively for communicability in the NFC communication. The detection signal is information of a connected state of the communication connector 93 of the endoscope 91 with the receptacle connector 112 of the processing apparatus 92. Note that a sequence of enabling the near field communication interfaces 106 and 107 with the interface controller 105a is predetermined. The near field communication interface 106 is enabled at first, and the near field communication interface 107 is enabled next.

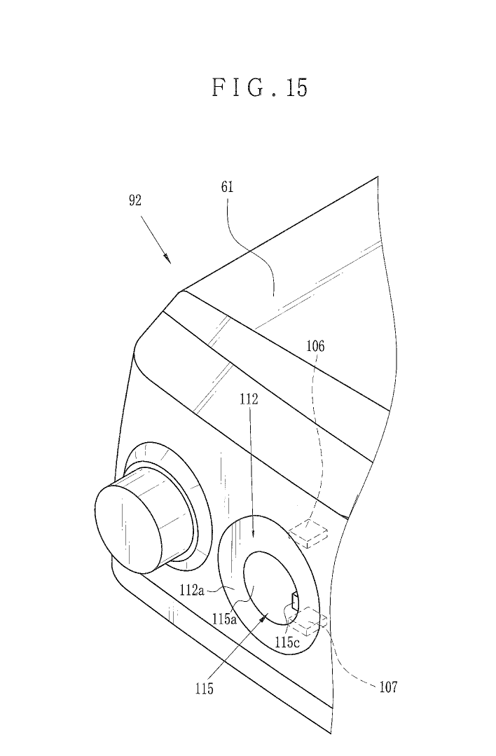

In FIGS. 13 and 15, the receptacle connector 112 is disposed on the apparatus housing 61 of the processing apparatus 92. The receptacle connector 112 includes a socket housing 113 and a shielding device 114 (shield wall) for the socket housing 113. The receptacle connector 112 has a cylindrical form, and has a front end 112a. The receiving cavity 115 is formed at the center to extend to the inside from the front end 112a.

The receiving cavity 115 is cylindrical and concentric with an outer surface of the receptacle connector 112, and includes an inner surface 115a, a flat receiving surface 115b and the key projection 115c. The flat receiving surface 115b is parallel with the front end 112a of the receptacle connector 112. The key projection 115c projects internally from the inner surface 115a.

The near field communication interfaces 106 and 107 are incorporated in the socket housing 113. The socket housing 113 is formed from a radio-transmissive material, for example, plastic material. The near field communication interfaces 106 and 107 are disposed to contact an inner wall of the socket housing 63 and in an inner space under the inner surface 115a. The near field communication interfaces 106 and 107 are disposed in a rotationally symmetric manner with 180 degrees about the center of the receiving cavity 115. A relative position of the near field communication interfaces 106 and 107 and the key projection 115c corresponds to a relative position of the near field communication interfaces 94 and 95 and the key way 101b. Engagement of the key way 101b with the key projection 115c positions the plug unit 101 and the receiving cavity 115 in the circumferential direction and the axial direction exactly, to position the near field communication interface 94 to the near field communication interface 106 and position the near field communication interface 95 to the near field communication interface 107.

The shielding device 114 covers an outer surface and distal end of the socket housing 113, and also extends to the surface of the receiving cavity 115. Openings 114a and 114b are formed in the shielding device 114 and disposed in the inner surface 115a of the receiving cavity 115. The openings 114a and 114b are aligned with respectively the near field communication interfaces 106 and 107 in the axial direction and circumferential direction of the receiving cavity 115. An inner diameter of the inner surface 115a of the receiving cavity 115 is determined according to the outer surface 101a of the plug unit 101 of the communication connector 93, to receive the outer surface 101a on the inner surface 115a.

The shielding device 114 is formed from the same material as the shielding device 103, and blocks radio waves. Only a component of the radio waves passed through the openings 114a and 114b can be transmitted externally in the radio waves originally emitted by the near field communication interfaces 106 and 107. Only a component of the radio waves passed through the openings 114a and 114b can be received by the near field communication interfaces 106 and 107 in the radio waves entered from the outside.

The near field communication interfaces 94, 95, 106 and 107 are an interface device according to the NFC communication similar to the first embodiment. The near field communication interfaces 94, 95, 106 and 107 become communicable upon being located at a distance of 1-3 cm from one another. The NFC communication is automatically started in case the near field communication interfaces 94, 95, 106 and 107 are all enabled and also located near to one another. Inside the receptacle connector 112, the near field communication interface 107 is opposed to the near field communication interface 106 through the openings 114a and 114b. Note that the near field communication interfaces 106 and 107 can be disposed in an area without communicability of radio waves, namely, at a distance more than 3 cm. In the present embodiment, the near field communication interfaces 106 and 107 are disposed at such a long distance as 4-8 cm. Therefore, no transmission or reception of radio waves occurs between the near field communication interfaces 106 and 107.

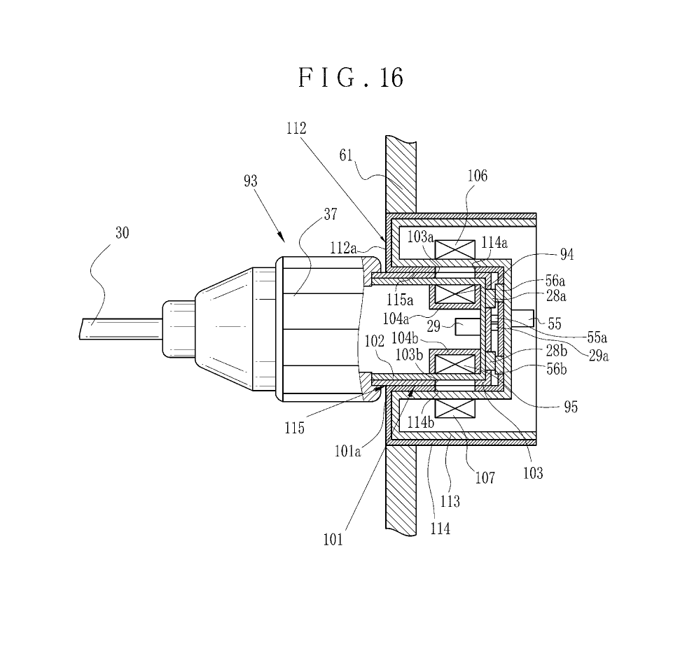

In FIG. 16, the communication connector 93 and the receptacle connector 112 are positioned by the key way 101b and the key projection 115c in the circumferential direction and the axial direction. As the outer surface 101a of the plug unit 101 is engaged with the inner surface 115a of the receiving cavity 115 to couple the communication connector 93 with the receptacle connector 112 tightly without drop, the near field communication interface 106 becomes opposed to the near field communication interface 94, and the near field communication interface 107 becomes opposed to the near field communication interface 95. A distance between the near field communication interfaces 94 and 106 and between the near field communication interfaces 95 and 107 is 1-3 cm, and sufficiently short for the NFC communication. As described above, the interface controller 98a successively enables the near field communication interfaces 94 and 95 upon detecting the connected state between the communication connector 93 and the receptacle connector 112. The interface controller 105a successively enables the near field communication interfaces 106 and 107. It is possible to establish the NFC communication by a first communication path 160 of the near field communication interfaces 94 and 106 and a second communication path 162 of the near field communication interfaces 95 and 107. Thus, an image signal is transmitted by the endoscope 91 to the processing apparatus 92, which transmits a control signal to the endoscope 91.

As described above, the openings 103a, 103b, 114a and 114b are formed in the shielding devices 103 and 114. In case the communication connector 93 is coupled with the receptacle connector 112 tightly without drop, the opening 103a is aligned with the opening 114a to oppose the near field communication interface 94 to the near field communication interface 106. The opening 103b is aligned with the opening 114b to oppose the near field communication interface 95 to the near field communication interface 107. Therefore, components of radio waves different from those passing the openings 103a, 103b, 114a and 114b are blocked by the shielding devices 103 and 114 while the NFC communication is established between the near field communication interfaces 94 and 106 and between the near field communication interfaces 95 and 107.

The operation of the endoscope system 90 is described now. To couple the communication connector 93 of the endoscope 91 to the receptacle connector 112 of the processing apparatus 92, the key projection 115c is aligned with the key way 101b. The outer surface 101a of the plug unit 101 is engaged with the inner surface 115a of the receiving cavity 115 to couple the communication connector 93 to the receptacle connector 112. Then the near field communication interface 94 is located close to the near field communication interface 106. The near field communication interface 95 is located close to the near field communication interface 107. The contact terminal 28a contacts the contact terminal 56a, the contact terminal 28b contacting the contact terminal 56b. The power supply for the processing apparatus 92 is turned on to supply the various elements with power in the processing apparatus 92. The powering device 35 in the endoscope 91 is also supplied with power through the contact terminals 28a, 28b, 56a and 56b. Elements in the endoscope 91 are also supplied with power by the powering device 35.

As the power supply for the processing apparatus 12 is turned on and the communication connector 93 is coupled to the receptacle connector 112, the connection detection devices 29 and 55 are turned on, to transmit detection signals to the CPUs 98 and 105. Then the CPU 98 successively enables the near field communication interfaces 94 and 95. The CPU 105 successively enables the near field communication interfaces 106 and 107. The first communication path 160 of the near field communication interfaces 94 and 106 becomes available for the NFC communication, the second communication path 162 of the near field communication interfaces and 107 becoming available simultaneously for the NFC communication, because of their short distance. For a sequence of enabling, the first communication path 160 is enabled before the second communication path 162 is enabled. Thus, the first communication path 160 becomes available for communication before the second communication path 162 does.

Divided packet data divided by the data division/restoration unit 108 are transmitted from the processing apparatus 92 to the endoscope 91 by the first communication path 160 of the near field communication interfaces 94 and 106 and the second communication path 162 of the near field communication interfaces 95 and 107. The CCD image sensor 18 is driven according to the control signal formed by restoring the divided packet data in the data division/restoration unit 97. The CCD image sensor 18 forms an image of a large intestine, and outputs an image signal. The image signal is transmitted to the processing apparatus 12 by the first and second communication paths 160 and 162 by way of divided packet data divided by the data division/restoration unit 97. The image signal restored by the data division/restoration unit 108 from the divided packet data is input to the digital signal processor 51, and processed in various functions of the image processing, before the image is displayed in the display panel 14. A physician or user can view the large intestine by observing the display panel 14.

As described heretofore, coupling of the communication connector 93 to the receptacle connector 112 starts the NFC communication through the first communication path 160 of the near field communication interfaces 94 and 106 and through the second communication path 162 of the near field communication interfaces 95 and 107. An image signal or control signal can be transmitted reliably between the endoscope 91 and the processing apparatus 92 even with data of a large size. The CCD image sensor 18 can function reliably to form an endoscopic image. The connected state between the communication connector 93 and the receptacle connector 112 is detected to establish the NFC communication upon enabling both of the first and second communication paths 160 and 162 of the near field communication interfaces 94 and 106, and 95 and 107. It is possible to prevent a problem of electric noise due to crosstalk of radio waves, and keep security in the transmission.

Owing to the NFC communication with the near field communication interfaces 94, 95, 106 and 107, it is possible to reduce laborious operation of a physician or user, as no complicated setting of the wireless LAN standards or the like is required. In a manner similar to the first embodiment, no shielding structure for a large area of the endoscope 91 and the processing apparatus 92 is required, so that a manufacturing cost can be reduced. Also, the surfaces of the connector housing 102 and the socket housing 113 are covered by the shielding devices 103 and 114 for containing the near field communication interfaces 94, 95, 106 and 107. Thus, it is possible to prevent a problem of electric noise due to crosstalk, and keep security in the transmission.

In the second embodiment, the near field communication interfaces 94, 95, 106 and 107 as two communication paths are used. However, three or more communication paths (or pairs) of near field communication interfaces can be used for the NFC communication. To this end, a communication connector can have near field communication interfaces with a respective angular range of 120 degrees. In a receptacle connector, near field communication interfaces can be disposed in correspondence with the near field communication interfaces of the communication connector.

In the second embodiment, it is possible to dispose a shielding cover on each of the communication connector 93 and the receptacle connector 112 in a similar manner to the variant of the first embodiment in FIGS. 7-10. The shielding cover can be set in a closed position for covering corresponding portions of the near field communication interfaces 94 and 106 and of the near field communication interfaces 95 and 107 to block radio waves, and shifted in an open position offset from the closed position for allowing transmission and reception of radio waves upon coupling between the communication connector 93 and the receptacle connector 112. For this structure, a shielding cover can be used for each of the near field communication interfaces, and openings corresponding to the shielding cover can be formed in shielding devices for the communication connector 93 and the receptacle connector 112.

Note that it is possible suitably to predetermine a sequence for enabling the near field communication interfaces 94, 95, 106 and 107, for the purpose of sequentially enabling the first and second communication paths 160 and 162. For example, the near field communication interfaces 94, 106 and 107 can be enabled initially at time points as desired, before the near field communication interface 95 can be enabled finally. Also, the near field communication interfaces 94, 95 and 106 can be enabled initially at time points as desired, before the near field communication interface 107 can be enabled finally. Furthermore, the near field communication interfaces 94 and 106 can be enabled initially at time points as desired, before the near field communication interfaces 95 and 107 can be simultaneously enabled finally.

### Third Embodiment

In the first embodiment, the NFC communication is started automatically upon enabling the near field communication interfaces. In contrast, the NFC communication in the third embodiment is authorized to start by a signal. Elements similar to those of the above embodiments are designated with identical reference numerals.

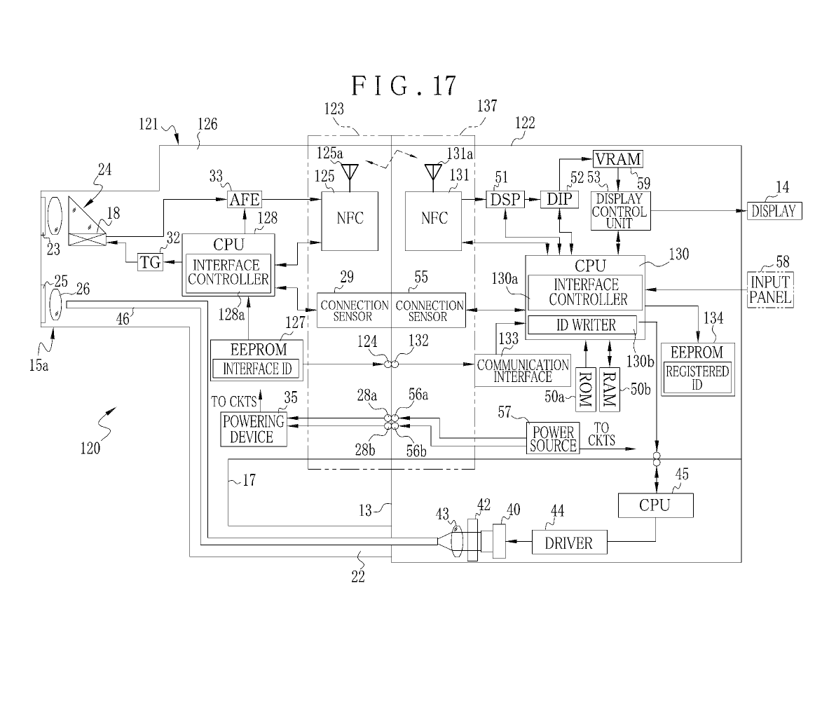

In FIG. 17, an endoscope system 120 includes an electronic endoscope 121 and a processing apparatus 122. A communication connector 123 (first connector) for the processing apparatus 12 includes a contact terminal 124, a near field communication interface 125 or NFC interface (first interface), the contact terminals 28a and 28b and the connection detection device 29. A grip handle 126 has the timing generator 32, the analog signal processor 33, the powering device 35, an EEPROM 127 and a CPU 128.

The EEPROM 127 is connected to the contact terminal 124 and the CPU 128, and stores an interface ID of the near field communication interface 125. An example of the interface ID is attribute information (interface information) of the near field communication interface 27, for example, a manufacturing serial number of the near field communication interface 125.

The near field communication interface 125 modulates the image signal from the analog signal processor 33 and converts this into radio signal in a manner similar to the near field communication interface 27 of the first embodiment. An antenna 125a is caused by the near field communication interface 125 to emit radio waves. The near field communication interface 125 demodulates a radio signal upon receiving radio waves from the processing apparatus 12 with the antenna 125a, and inputs a control signal to the CPU 128. Also, an interface controller 128a in the CPU 128 enables the near field communication interface 125, which outputs an interface ID, as will be described later.

In case a detection signal is input by the connection detection device 29, the interface controller 128a enables the near field communication interface 125 for communicability in the NFC communication. The detection signal is information of a connected state of the communication connector 123 of the endoscope 121 with a receptacle connector 137 (second connector) or socket device of the processing apparatus 122.

Upon enabling the near field communication interface 125, the CPU 128 causes the near field communication interface 125 to transmit the interface ID read from the EEPROM 127. The transmission of the interface ID with the near field communication interface 125 is continued until a control signal is generated by the processing apparatus 122.

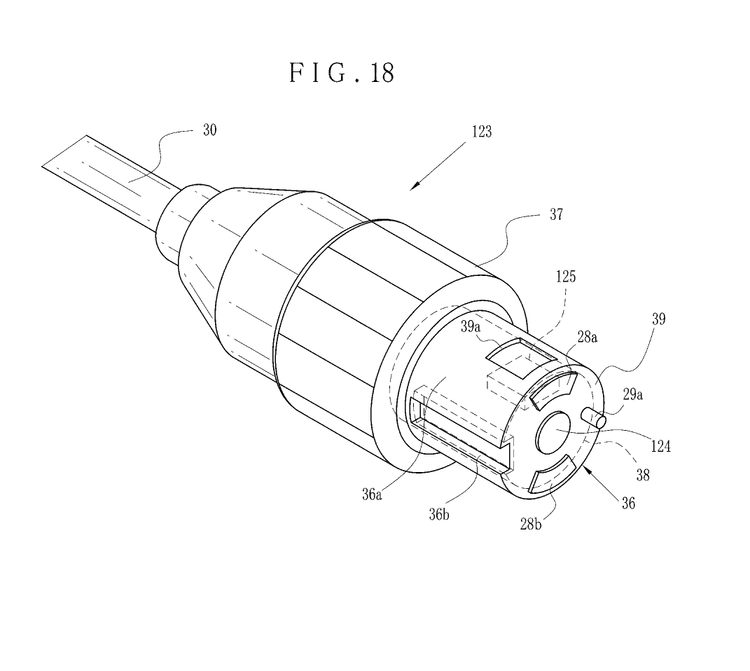

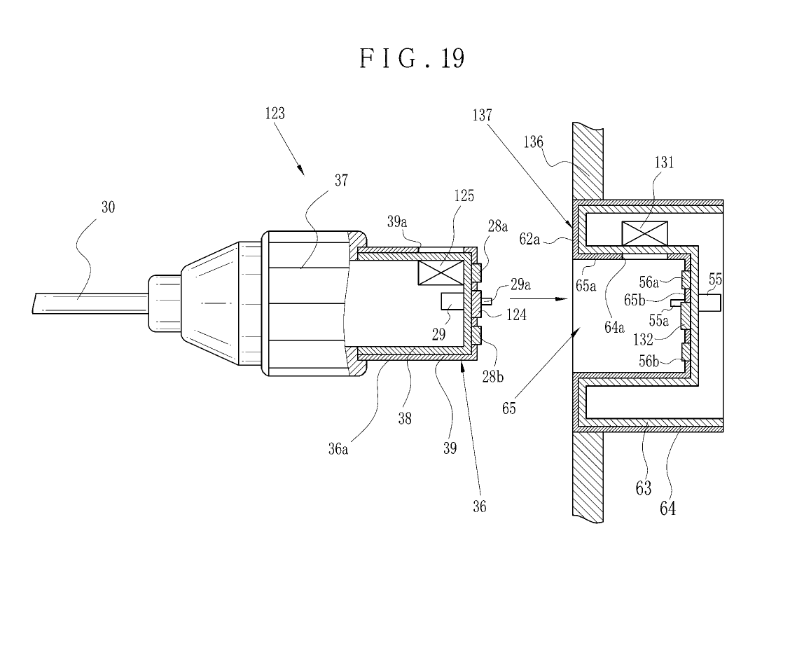

In FIGS. 18 and 19, the contact terminal 124 is disposed on the plug unit 36 in the communication connector 123. Note that lines or wires are not depicted in FIG. 19 for connecting the contact terminal 124 to the EEPROM 127. The contact terminal 124 is disposed at the center of the proximal end of the plug unit 36.

The near field communication interface 125 is contained in the connector housing 38 in a manner similar to the near field communication interface 27 of the first embodiment, is disposed in an inner space under the outer surface 36a, and contacts an inner wall of the connector housing 38. The opening 39a is aligned with the near field communication interface 125.

The processing apparatus 122 includes a CPU 130 (information acquisition device), the digital signal processor 51, the digital image processor 52, the display control unit 53, a near field communication interface 131 or NFC interface (second interface), the connection detection device 55, the contact terminals 56a and 56b, the power source 57, the input panel 58, a contact terminal 132, a communication interface 133 (information acquisition device) and an EEPROM 134 (authorizer).

In case the communication interface 133 becomes connected to the EEPROM 127 by the contact terminals 124 and 132, the communication interface 133 reads an interface ID of the near field communication interface 125 and transmits this to the CPU 130. An example of the communication interface 133 is a serial communication interface or the like which is constructed to read the interface ID from the EEPROM 127 of the endoscope 121 on-line for transmission to the CPU 130.

The CPU 130 includes an interface controller 130a (authorizer) and an ID writer 130b for registration. The ID writer 130b writes an interface ID of the near field communication interface 125 from the communication interface 133 to the EEPROM 134 as registered ID (authorization information) of the endoscope 121. Assuming that there is registered ID stored in the EEPROM 134, it is possible in the ID writer 130b to overwrite the registered ID by rewriting of old registered ID, or to write the new registered ID additionally without deleting old registered ID.

In case a detection signal for a connected state between the communication connector 123 of the endoscope 121 and the receptacle connector 137 of the processing apparatus 122 is input to the interface controller 130a by the connection detection device 55, the interface controller 130a performs control to enable the near field communication interface 131 for communicability.

In case both of the near field communication interfaces 125 and 131 are enabled and also located close to one another according to the present embodiment, at first the near field communication interface 27 transmits an interface ID. Thus, the near field communication interface 54 receives the interface ID and transmits this to the CPU 130.

The interface controller 130a enables the near field communication interface 131 and monitors an interface ID received by the near field communication interface 131. The interface controller 130a authorizes the NFC communication only in case the received interface ID is identical with the registered ID (authorization information) in the EEPROM 134. In response to authorization of the NFC communication in the interface controller 130a, the CPU 130 controls the near field communication interface 131 to start transmitting a control signal, and start receiving an image signal.

In FIG. 19, an apparatus housing 136 of the processing apparatus 122 has the receptacle connector 137, which includes the socket housing 63 and the shielding device 64 for covering the socket housing 63 in a manner similar to the receptacle connector 62 of the first embodiment.

The contact terminal 132 is disposed in the receiving cavity 65 of the receptacle connector 137. The contact terminal 132 is positioned at the center of the flat receiving surface 65b of the receiving cavity 65. In case the communication connector 123 is coupled with the receptacle connector 137 tightly without drop, the contact terminal 132 contacts the contact terminal 124. Thus, the communication interface 133 is ready to read out an interface ID from the EEPROM 127 through the contact terminals 124 and 132.

An antenna 131a is provided in the near field communication interface 131, which is contained in the socket housing 63 in a manner similar to the near field communication interface 54 described above. The near field communication interface 131 contacts an inner surface of the socket housing 63 and disposed in an inner space under the inner surface 65a. The opening 64a is formed in a portion aligned with the near field communication interface 131.

The operation of the endoscope system 120 is described. To couple the communication connector 123 of the endoscope 121 to the receptacle connector 137 of the processing apparatus 122, the key projection 65c is aligned with the key way 36b. The outer surface 36a of the plug unit 36 is received on the inner surface 65a of the receiving cavity 65 to couple the communication connector 123 to the receptacle connector 137 tightly without drop. Consequently, the near field communication interface 125 is located close to the near field communication interface 131. The contact terminal 28a contacts the contact terminal 56a, the contact terminal 28b contacting the contact terminal 56b. The contact terminal 124 contacts the contact terminal 132.

In case the power supply for the processing apparatus 122 is turned on and the communication connector 123 becomes coupled with the receptacle connector 137, the communication interface 133 reads an interface ID from the EEPROM 127 through the contact terminals 124 and 132, and transmits this to the CPU 130. The ID writer 130b of the CPU 130 writes the received interface ID to the EEPROM 134 as registered ID. The connection detection devices 29 and 55 are turned on and supply detection signals to the CPUs 128 and 130. The CPU 128 enables the near field communication interface 125. The CPU 130 enables the near field communication interface 131.

The near field communication interfaces 125 and 131 enabled simultaneously transmit the interface ID to the CPU 130 in the processing apparatus 122. Assuming that the interface ID is found equal to the registered ID in the EEPROM 134, then the interface controller 130a authorizes the NFC communication, and controls the near field communication interface 131 to start transmitting a control signal and start receiving an image signal. The CCD image sensor 18 is driven according to the control signal received by the endoscope 121 from the processing apparatus 122 through the near field communication interfaces 125 and 131. The CCD image sensor 18 images an intestinal wall and outputs an image signal. The image signal is input to the processing apparatus 122 by the near field communication interfaces 125 and 131, and processed in various functions of the image processing, before the display panel 14 displays an image. A physician or user can view the display panel 14 to diagnose the intestinal wall.

As described heretofore, the interface ID is registered on the side of the processing apparatus 122 upon coupling the communication connector 123 to the receptacle connector 137. The near field communication interface 131 of the processing apparatus 122 is communicable only with the near field communication interface 125 of the equality of the registered ID. Therefore, it is possible to prevent the NFC communication with an interface device other than the near field communication interface 125 of the endoscope 121 connected with the processing apparatus 122. This is effective in preventing a problem of electric noise due to crosstalk of radio waves, and keeping security in the transmission, in addition to the effect of the first embodiment.

In the present embodiment, it is checked whether the interface ID coincides with the registered ID. However, authorization information in a form different from the interface ID can be used in place of the registered ID, for example, a security code, alphanumeric expression or the like specialized for authorization. The authorization information is registered in the memory. It is checked whether the interface ID corresponds to the authorization information, so that the NFC communication can be authorized upon detecting the correspondence.

### Fourth Embodiment

In a fourth embodiment, the plural near field communication interfaces of the second embodiment are repeated unlike the third embodiment. Elements similar to those of the above embodiments are designated with identical reference numerals.

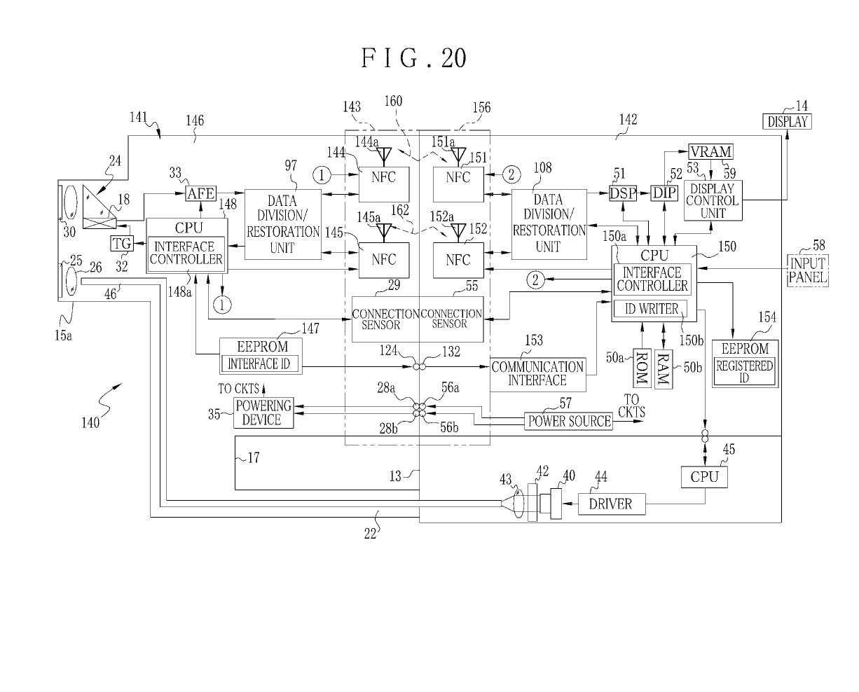

In FIG. 20, an endoscope system 140 includes an electronic endoscope 141 and a processing apparatus 142. A communication connector 143 (first connector) includes the contact terminal 124, two near field communication interfaces 144 and 145 or NFC interfaces (first interfaces), the contact terminals 28a and 28b and the connection detection device 29. A grip handle 146 includes the timing generator 32, the analog signal processor 33, the powering device 35, the data division/restoration unit 97, an EEPROM 147 and a CPU 148.

Each of the near field communication interfaces 144 and 145, similar to the near field communication interfaces 94 and 95 of the second embodiment, modulates divided packet data from the data division/restoration unit 97, converts this into a radio signal, causes the antenna 94a or 95a to emit radio waves of the radio signal, and then receives radio waves from the processing apparatus 122 by use of the antenna 94a or 95a, demodulates those into divided packet data, and inputs this to the data division/restoration unit 97.

The EEPROM 147 is connected to the contact terminal 124 and the CPU 148, and stores an interface ID of the near field communication interfaces 144 and 145. An example of the interface ID is attribute information (interface information) of the near field communication interfaces 144 and 145, for example, a manufacturing serial number of the near field communication interfaces 144 and 145. Note that a sequence of readout of the interface ID of the near field communication interfaces 144 and 145 with a communication interface 153 (information acquisition device) is predetermined. The interface ID of the near field communication interface 144 is read out at first, and the interface ID of the near field communication interface 145 is read out next.

An interface controller 148a is incorporated in the CPU 148. In case a detection signal is input by the connection detection device 29, the interface controller 148a enables the near field communication interfaces 144 and 145 for communicability in the NFC communication. The detection signal is information of a connected state of the communication connector 143 of the endoscope 141 with a receptacle connector 156 (second connector) or socket device of the processing apparatus 142.

Upon enabling the near field communication interfaces 144 and 145, the CPU 148 causes those to transmit interface IDs read from the EEPROM 147. The near field communication interface 144 transmits only the interface ID of the near field communication interface 144. The near field communication interface 145 transmits only the interface ID of the near field communication interface 145. The transmission of their interface IDs is continued until a control signal is generated by the processing apparatus 142.

For the communication connector 143, the communication connector 93 of the second embodiment is repeated but with differences in the contact terminal 124 and the near field communication interfaces 144 and 145, which are internally arranged in a manner similar to the near field communication interfaces 94 and 95 of the second embodiment. The contact terminal 124 is disposed at the center of the proximal end in a manner similar to the communication connector 123 of the third embodiment.

The processing apparatus 142 includes a CPU 150 (information acquisition device), the digital signal processor 51, the digital image processor 52, the display control unit 53, near field communication interfaces 151 and 152 or NFC interfaces (second interfaces), the data division/restoration unit 108, the connection detection device 55, the contact terminals 56a and 56b, the power source 57, the input panel 58, the contact terminal 132, the communication interface 153 and an EEPROM 154 (authorizer). In the receptacle connector 156 of the processing apparatus 142 are disposed the near field communication interfaces 151 and 152, the contact terminal 132 and the contact terminals 56a and 56b.

The near field communication interfaces 151 and 152 modulate packet data divided by the data division/restoration unit 108 into a radio signal, which antennas 151a and 152a transmit by way of radio waves. Also, the radio waves emitted by the near field communication interfaces 144 and 145 of the endoscope 141 are received by the antennas 151a and 152a, from which a radio signal is demodulated and input to the data division/restoration unit 108 as divided packet data.

The communication interface 153 upon connection to the EEPROM 147 through the contact terminals 124 and 132 reads an interface ID of the near field communication interfaces 144 and 145 and transmits this to the CPU 150. An example of the communication interface 153 is a serial communication interface similar to the communication interface 133 of the third embodiment.

The CPU 150 includes an interface controller 150a (authorizer) and an ID writer 150b for registration. The ID writer 150b writes the interface IDs of the near field communication interfaces 144 and 145 from the communication interface 153 to the EEPROM 154 as registered ID (authorization information) of the endoscope 141. It is possible in the ID writer 150b to overwrite the registered ID by rewriting of old registered ID in a manner similar to the ID writer 130b of the third embodiment, or to write the new registered ID additionally without deleting old registered ID.

The ID writer 150b writes the interface IDs in a suitable sequence, for example a sequence of readout from the EEPROM 147 of the endoscope 141 with the communication interface 153, so that the NFC communication is possible between the near field communication interfaces 144 and 145 and between the near field communication interfaces 151 and 152. The interface controller 150a authorizes the NFC communication upon receiving the interface IDs through the first communication path 160 of the near field communication interfaces 144 and 151 and the second communication path 162 of the near field communication interfaces 145 and 152 according to the sequence of writing of the interface IDs.

In case a detection signal is input by the connection detection device 55, the interface controller 150a enables the near field communication interfaces 151 and 152 for communicability in the NFC communication. The detection signal is information of a connected state of the communication interface 153 of the endoscope 141 with the receptacle connector 156 of the processing apparatus 142.

In case the first communication path 160 of the near field communication interfaces 144 and 151 is enabled and set at the short distance and the second communication path 162 of the near field communication interfaces 145 and 152 is enabled and set at the short distance, then the near field communication interfaces 151 and 152 receive the interface IDs and input those to the CPU 150, because the near field communication interfaces 144 and 145 initially output their interface IDs.

The interface controller 150a enables the near field communication interfaces 151 and 152 and monitors an interface IDs received by the near field communication interfaces 151 and 152. The interface controller 150a authorizes the NFC communication only in case the received interface IDs are identical with the registered ID in the EEPROM 154 and in case the interface IDs of the near field communication interfaces 144 and 145 are received through the first and second communication paths 160 and 162 of the near field communication interfaces 144 and 151 and of the near field communication interfaces 145 and 152.

For the receptacle connector 156, the receptacle connector 112 of the second embodiment is repeated but with differences in having the contact terminal 132 and the near field communication interfaces 151 and 152. The near field communication interfaces 151 and 152 are contained in the arrangement similar to the near field communication interfaces 106 and 107 described above. In case the communication connector 143 is coupled to the receptacle connector 156 tightly without drop, the contact terminal 132 becomes disposed for contacting the contact terminal 124 in a manner similar to the receptacle connector 137 of the third embodiment.

The operation of the endoscope system 140 is described. The communication connector 143 is coupled with the receptacle connector 156 in a manner similar to the second embodiment. The near field communication interface 144 is located close to the near field communication interface 151. The near field communication interface 145 is located close to the near field communication interface 152. The contact terminal 28a contacts the contact terminal 56a, the contact terminal 28b contacting the contact terminal 56b. The contact terminal 124 contacts the contact terminal 132.

In case the power supply for the processing apparatus 142 is turned on and the communication connector 143 becomes coupled to the receptacle connector 156, then the communication interface 153 reads the interface IDs of the near field communication interfaces 144 and 145 from the EEPROM 147 through the contact terminals 124 and 132, and transmits those to the CPU 150. The ID writer 150b of the CPU 150 writes the interface IDs to the EEPROM 154 in the predetermined sequence as registered ID. The connection detection devices 29 and 55 are turned on to supply the CPUs 148 and 150 with detection signals. The CPU 148 enables the near field communication interfaces 144 and 145. The CPU 150 enables the near field communication interfaces 151 and 152.

The interface ID of the near field communication interfaces 144 and 145 is transmitted to the CPU 150 on the side of the processing apparatus 142 through the first communication path 160 of the near field communication interfaces 144 and 151 and the second communication path 162 of the near field communication interfaces 145 and 152 after being enabled. The interface controller 150a authorizes the NFC communication only in case the received interface ID is identical with the registered ID and in case the interface ID of the near field communication interfaces 144 and 145 is received through the first and second communication paths 160 and 162 of the near field communication interfaces 144 and 151 and of the near field communication interfaces 145 and 152.

The processing apparatus 142 transmits the divided packet data from the data division/restoration unit 108 to the endoscope 141 through the first communication path 160 of the near field communication interfaces 144 and 151 and the second communication path 162 of the near field communication interfaces 145 and 152. A control signal is restored by the data division/restoration unit 97 according to the divided packet data, so as to drive the CCD image sensor 18 by the control signal. The CCD image sensor 18 images an object of the intestinal wall to output an image signal. The CCD image sensor 18 transmits the image signal to the processing apparatus 142 by way of divided packet data from the data division/restoration unit 97 through the first and second communication paths 160 and 162 of the near field communication interfaces 144 and 151, and 145 and 152. The image signal restored by the data division/restoration unit 108 according to the divided packet data is input to the digital signal processor 51, and processed in the image processing of various functions, so that an endoscopic image is displayed on the display panel 14. A physician or user views the intestinal wall with the display panel 14.

In the above embodiments, the distance between the near field communication interfaces at which those are communicable with each other is as short as 1-3 cm. However, a distance for the communicability between the near field communication interfaces can be shorter than 1 cm or longer than 3 cm.

In the above embodiments, the connection detection devices 29 and 55 are mechanical switches. However, other sensors can be used for the detecting connection, for example, photo interrupter or optical sensor. Furthermore, a detection circuit can be added by way of a connection detection device for monitoring supplied power of the power source, and detecting an event of power supply from the processing apparatus to the endoscope 11 to generate a detection signal.

In the above embodiment, the light source connector is distinct from the communication connector. However, the light source connector can constitute the communication connector typically in case the light source apparatus and the processing apparatus are included in a single composite apparatus. The feature of the above embodiments can be used in the light source connector for use as the communication connector. In the above embodiment, the LED is used for the light source 40. However, other light sources can be used as the light source 40, for example, a xenon lamp and the like.

In the above embodiment, the endoscope is for the medical use. However, an endoscope of the invention may be a probe, and can be used for an industrial purpose.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An endoscope system including an endoscope having a tip device for entry in a body cavity, and an imaging unit for receiving light from an object in said body cavity through said tip device to output an image signal, and a processing apparatus for forming an endoscopic image according to said image signal from said imaging unit, said endoscope system comprising:
a first connector disposed on a proximal end side of said endoscope;
a second connector disposed on said processing apparatus, and coupled with said first connector;
a first near field communication interface, disposed in said first connector, for wirelessly transmitting said image signal from said imaging unit, and wirelessly receiving a control signal for controlling said imaging unit;
a second near field communication interface, disposed in said second connector, and positioned at a short distance to said first near field communication interface upon setting of said first and second connectors in a connected state;
a connection detection device for detecting said connected state between said first and second connectors;
an interface controller for controlling said first and second near field communication interfaces for near field wireless communication in case said connection detection device detects said connected state between said first and second connectors.

2. An endoscope system as defined in claim 1, wherein said first and second near field communication interfaces perform said near field wireless communication upon being enabled and also located at a short distance for communicability.

3. An endoscope system as defined in claim 1, wherein in case said connection detection device detects said connected state between said first and second connectors, said interface controller enables said first near field communication interface.

4. An endoscope system as defined in claim 1, wherein in case said connection detection device detects said connected state between said first and second connectors, said interface controller enables said second near field communication interface.

5. An endoscope system as defined in claim 1, further comprising a contact terminal, disposed in said first and second connectors, for supplying said endoscope with power from said processing apparatus upon setting of said first and second connectors in said connected state;
wherein said interface controller enables said first near field communication interface after starting supplying said endoscope with power through said contact terminal.

6. An endoscope system as defined in claim 1, further comprising:
a shielding device for covering said first and second connectors to block radio waves;
an opening, formed in said shielding device, and disposed between said first and second near field communication interfaces opposed to one another while said first and second connectors are in said connected state.

7. An endoscope system as defined in claim 6, wherein at least one of said first and second connectors include a shielding cover disposed movably relative to said opening between open and closed positions, for closing said opening aligned with said first and second near field communication interfaces upon being set in said closed position, and for opening said opening upon being set in said open position.

8. An endoscope system as defined in claim 6, wherein said first connector includes a plug unit, disposed to project toward said processing apparatus, for containing said first near field communication interface;
said second connector includes a receiving cavity for engagement with said plug unit to set said first and second connectors in said connected state;
said second near field communication interface is disposed on an inner wall of said receiving cavity, and said shielding device covers a surface of said second connector disposed around said receiving cavity.

9. An endoscope system as defined in claim 1, wherein said second near field communication interface receives interface information of said first near field communication interface;
further comprising:
an information acquisition device for acquiring authorization information of said first near field communication interface through a path different from said second near field communication interface upon setting said first and second connectors in said connected state;
an authorizer for authorizing said near field wireless communication between said first and second near field communication interfaces in case said interface information corresponds to said authorization information.

10. An endoscope system as defined in claim 9, further comprising a memory, incorporated in said endoscope, for storing said authorization information;
wherein said information acquisition device is incorporated in said processing apparatus, and accesses said memory upon setting said first and second connectors in said connected state.

11. An endoscope system as defined in claim 1, comprising at least first and second communication paths, which are provided in parallel with one another between said first and second connectors, and each of which includes said first and second near field communication interfaces, for simultaneously performing said near field wireless communication upon setting of said first and second connectors in said connected state.

12. An endoscope system as defined in claim 11, wherein said second near field communication interface is so disposed that transmission of radio waves is blocked by a distance between said first and second communication paths.

13. An endoscope system as defined in claim 11, further comprising a separating wall, disposed with said first near field communication interface, for blocking radio waves between said first and second communication paths.

14. An endoscope system as defined in claim 11, wherein said second near field communication interface is enabled one after another in relation to said first and second communication paths upon setting of said first and second connectors in said connected state.

15. An endoscope system as defined in claim 11, wherein said first near field communication interface is enabled one after another in relation to said first and second communication paths upon setting of said first and second connectors in said connected state.

16. An endoscope system as defined in claim 11, wherein said endoscope includes a data division unit for dividing data of said image signal from said imaging unit into divided packet data, and transmitting said divided packet data to said first and second communication paths in distribution;
said processing apparatus includes a data restoration unit for receiving said divided packet data from said first and second communication paths, and restoring said image signal.

17. An endoscope system as defined in claim 11, wherein in relation to each of said first and second communication paths, said second near field communication interface receives interface information of said first near field communication interface;
further comprising:
an information acquisition device for acquiring authorization information of said first near field communication interface through a path different from said second near field communication interface upon setting said first and second connectors in said connected state;
an authorizer for authorizing said near field wireless communication between said first and second near field communication interfaces in case said interface information corresponds to said authorization information in relation to both of said first and second communication paths.

18. An endoscope system as defined in claim 1, further comprising a light source apparatus, connected with said endoscope, for generating light to illuminate said object;
wherein said first and second connectors function also for connection to said light source apparatus.
